(19) United States
(12) Patent Application Publication
(10) Pub. No.: US 2024/0374350 A1
(43) Pub. Date: Nov. 14, 2024

(54) SYSTEMS AND METHODS FOR SIMULATION OF ATTACHMENTS AND ALIGNERS

(71) Applicant: LOMA LINDA UNIVERSITY, Loma Linda, CA (US)

(72) Inventors: Roberto Savignano, Loma Linda, CA (US); Rodrigo F. Viecilli, Loma Linda, CA (US)

(73) Assignee: LOMA LINDA UNIVERSITY, Loma Linda, CA (US)

(21) Appl. No.: 17/997,178

(22) PCT Filed: Apr. 26, 2021
(86) PCT No.: PCT/US2021/070455

§ 371 Date: Oct. 26, 2022

Related U.S. Application Data
(60) Provisional application No. 63/015,592, filed on Apr. 26, 2020.

(51) Int. Cl. A61C 7/08; A61C 7/00; G06F 30/27

(52) U.S. Cl. CPC A61C 7/08; G06F 30/27; A61C 2007/004

(57) ABSTRACT

A processor-implemented system and method for digital aligner simulation include determining axial plane values for at least a group of teeth and a coordinate system. A crown location is determined using the coordinate system. An attachment at the crown location is determined for a digital aligner to be provided in a physical format for the group of teeth. A digital penetration is determined between the digital aligner and the tooth. At least a finite element analysis is applied to the model to determine the force system delivered to the tooth/teeth. The center of rotation, and axis of rotation and therefore the whole movement description is calculated. At least one variation to the attachment or the digital aligner is provided based in part on the orthodontic data.

### TECHNICAL FIELD

The present disclosure generally relates to dental attachments or aligners. In particular, the present disclosure relates to systems and methods for simulation to optimize the design of digital attachments and aligners that help to remove or reduce visits to a dental provider for extended periods during which a tooth or a group of teeth are moved or aligned to a desired position. As such, the present disclosure can provide aligner and attachments designs and modifications with realistic dentoalveolar simulations.

### BACKGROUND

Orthodontic aligners are generally used to align teeth for medical and cosmetic reasons. There is limited published science about an aligner's biomechanical interactions with teeth. The design of aligners and the choice of a best attachment for each movement for a tooth or a group of teeth, and for each patient may be based on trial-and-error, along with recommendations from orthodontic companies. The limited published scientific rationale and data makes it difficult to ascertain the number of visits required and the number of subsequent adjustments required to enable a level of comfort and a proper correction to the intended positions.

While there are software providing tests designs for aligners and attachments utilizing a transducer approaches, there is limited data published in the literature to biomechanically justify the approaches currently in effect. For example, a limitation of the transducer approaches is that realistic oral boundary conditions and deformations of the periodontal ligament on a target tooth, as well as on adjacent teeth, may not be considered in the transducer approaches. Transducer approaches further require a review of the current position of an aligner and attachments are studied in comparison with the initial position. The review also takes into consideration any jostling for space caused by movement of the target tooth and the adjacent teeth. As the aligner, in such a process, depends on interaction of a surface of the aligner with the teeth, transducer measurements in the transducer approaches tend to be rigid and somewhat misleading. For example, because the transducer approaches are rigid, they may be unable to simulate periodontal ligament deformations and take such issues into account during an initial visit to set the aligner into place. Furthermore, transducer approaches generally are only used for research purposes on sample dentition, rather than for specific patient treatments because they can require significant amounts of time and money for generation of models.

Currently there are also Finite Element Analysis models published, but these models do not take into account enough boundary conditions (occlusal load) and the comparison between different simulated design is mathematically weak. It is more qualitative comparison than a quantitative numerical comparison.

### SUMMARY

Provided here are systems and methods for simulating attachments and aligners for a tooth or a group of teeth that are moved or aligned to a desired position.

Certain embodiments include a system including at least one processor and memory including instructions that, when executed in part by the at least one processor, cause the at least one processor to support or perform certain function is disclosed. In addition, a method using the system or any system configured in the manner as disclosed herein is also discussed. Furthermore, a non-transitory medium including instructions for executing on at least one processor is available to enable any system to support or perform the functions as follows.

In some embodiments, an orthodontic device or system can deliver a complex force system which involves forces and moments in space, such as three forces and three moments in the 3D space. Orthodontic movement can be defined by a combination of moments and forces applied to a tooth. Teeth morphology can be different and/or asymmetric, such that the forces/moments can have a different impact on tooth movement for each plane and each tooth.

In some embodiments, a system, via the at least one processor, can determine axial plane values for at least one tooth or a group of teeth. A coordinate system can be determined from the axial plane values. A crown location, such as a crown center or other suitable crown location, can be located using the coordinate system. The crown location may be for the tooth or a tooth of the group of teeth. The tooth may be a target tooth that requires the most movement in an aligning process for a patient. An attachment at the crown location for a digital aligner can be determined. The attachment at the crown location for the digital aligner may be determined for provision of a physical format for the group of teeth. A digital penetration can be determined between the digital aligner and the tooth. A simulation model, such as including at least a finite element analysis or other suitable model, e.g., statistical models, supervised learning models, etc., can be applied to determine orthodontic data for a center of rotation for the tooth based at least in part on the digital penetration. At least one variation to the attachment or the digital aligner can be provided based in part on the orthodontic data.

In some embodiments, a method or process can include obtaining or receiving information related to sets of sets of teeth (e.g., 2 or more full sets of teeth), e.g., by combining cone beam computed tomography (CBCT) and optical scan for selected patients or more in general 3D digital reconstructions. Thereafter, a simulation model, e.g., including finite element analysis, statistical analysis, etc. for each tooth can evaluate the centers of rotation thereof. In one embodiment, a finite element analysis can be performed for various different force systems (e.g., up to 510 or more force systems) for each tooth to evaluate their centers of rotation. The center of rotation locations can be analyzed to show that the moment/force effect relates to a spatial plane on which the moment is applied, to the force direction and to the tooth morphology. The tooth dimensions on each plane can be used to derive their influence on tooth movement. Accordingly, qualified professionals, e.g., orthodontists, dental practitioners, etc., can determine how the teeth move and their axes of resistance, depending on their morphology alone. In some examples, movement can be controlled, determined, etc. by a parameter (“k”), which depends on tooth dimensions and force system features. The parameter k for a specific tooth can be generated, calculated, etc. using a CBCT and a specific set of predictors. In one example, parameter k can be defined as a constant of proportionality depending on a plane, force direction, and the specific tooth.

Still further, in some embodiments, statistical analyses can be performed to obtain/generated generalized formulae to locate the tooth C_{RES} and calculate an approximate “k” for every tooth and force system. For example, multiple linear regression analysis can be conducted to assess the influence of the teeth morphological data for at least one dataset. Hypotheses further can be tested at a prescribed level (e.g., an alpha=0.05 level) for at least one additional “k” dataset. Furthermore, to evaluate the performance of the regression models, predictors can be tested on a randomly chosen tooth, such as a mandibular central incisor reconstructed through combination of CBCT and an optical scanner. Then, C_{RES} coordinates and k values can be derived using predictors obtained by the regression models, and the FEA datasets can be compared.

Those skilled in the art will appreciate the above stated advantages and other advantages and benefits of various additional embodiments by reading the following detailed description of the embodiments with reference to the drawings.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain the principles of the embodiments discussed herein. No attempt is made to show structural details of this disclosure in more detail than may be necessary for a fundamental understanding of the exemplary embodiments discussed herein and the various ways in which they may be practiced. According to common practice, the various features of the drawings discussed below are not necessarily drawn to scale. Dimensions of various features and elements in the drawings may be expanded or reduced to more clearly illustrate the embodiments of the disclosure.

FIG. 1 is an example process flow in accordance with an aspect of the present disclosure.

FIG. 2A is one aspect of an example process flow for performing the present simulation of attachments and aligners in accordance with this disclosure.

FIG. 2B illustrates example dental features observed or determined for the present simulation of attachments and aligners in accordance with this disclosure.

FIG. 3A is another aspect of an example process flow for performing the present simulation of attachments and aligners in accordance with this disclosure.

FIG. 3B is an example step in the aspect of FIG. 3A of the present simulation of attachments and aligners in accordance with this disclosure.

FIG. 4 is yet another aspect of an example process flow for performing the present simulation of attachments and aligners in accordance with this disclosure.

FIG. 5 provides example aspects of alignment available via the example simulation of attachments and aligners in accordance with this disclosure.

FIGS. 6A and 6B are graphical representations of the torque quantity and torque quality obtained for different depths of attachments and aligners in accordance with this disclosure.

FIGS. 6C and 6D are graphical representations of the torque quantity and torque quality obtained for different diameters of attachments and aligners in accordance with this disclosure.

FIGS. 6E and 6F are graphical representations of the rotation quantity and rotation quality obtained for different depths of attachments and aligners in accordance with this disclosure.

FIGS. 6G and 6H are graphical representations of the rotation quantity and rotation quality obtained for different diameters of attachments and aligners in accordance with this disclosure.

FIGS. 61, 6J, and 6K are graphical representations of the deviation of the rotation axis, moment Z-axis, and the C_{ROT} deviation for different depths of attachments and aligners in accordance with this disclosure.

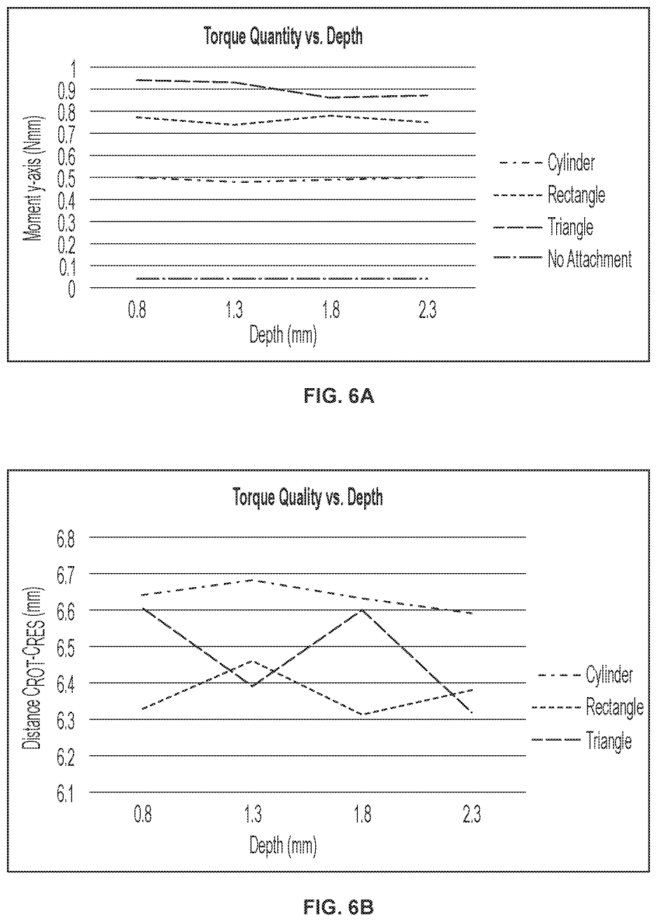

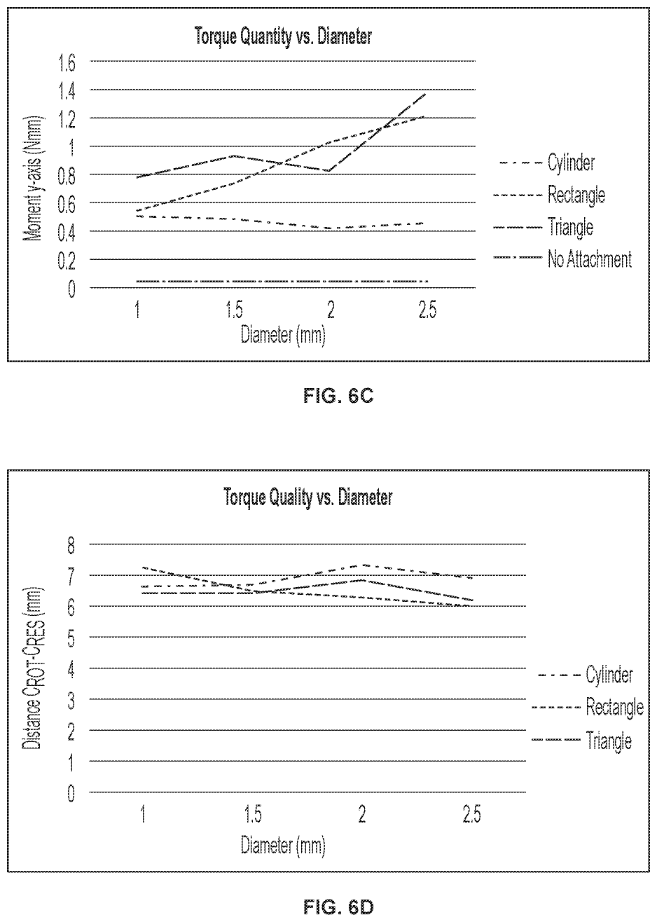

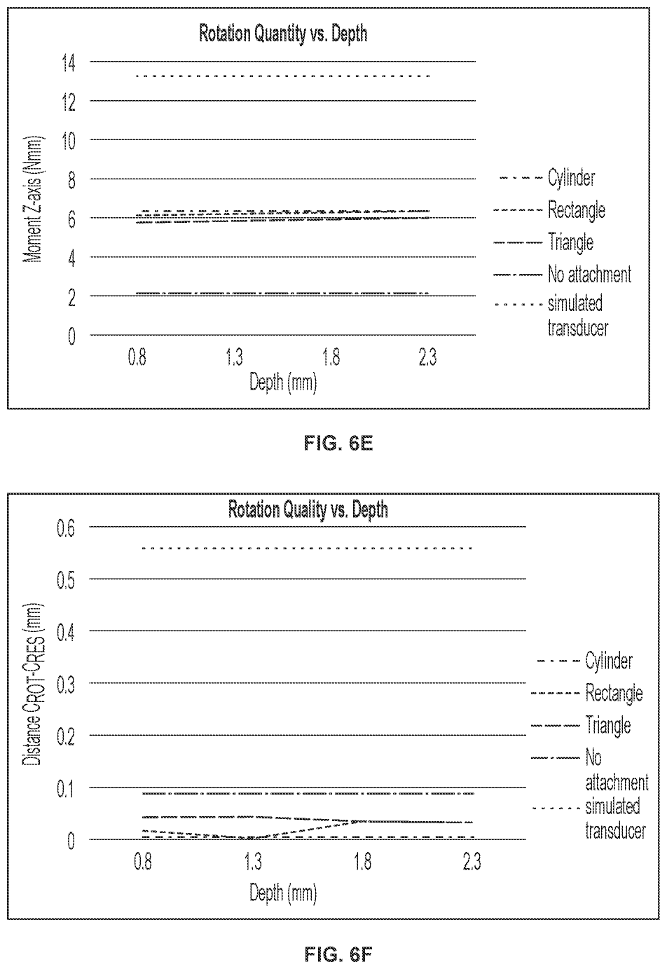

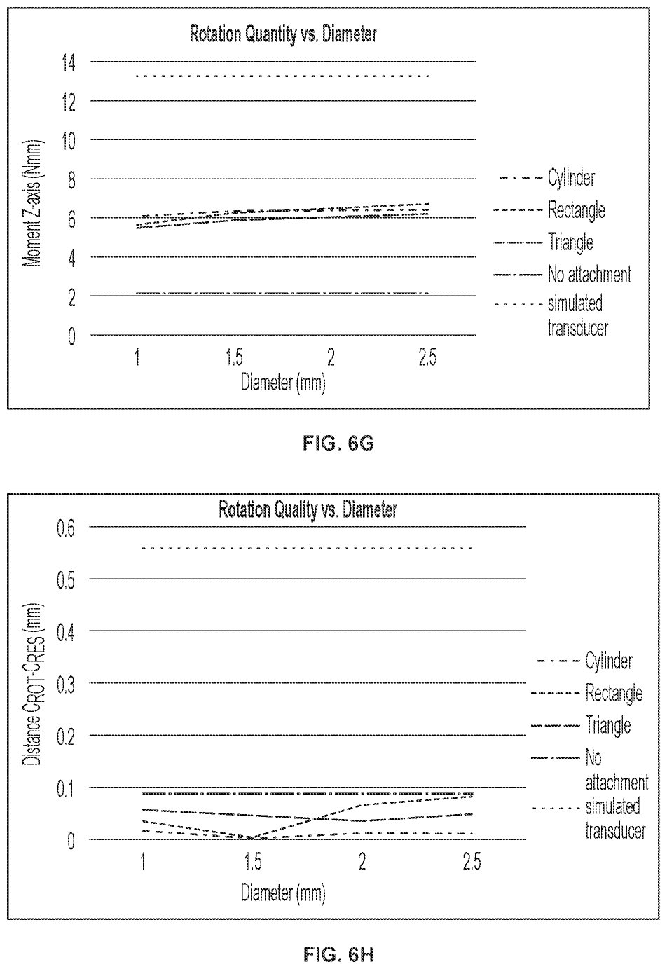

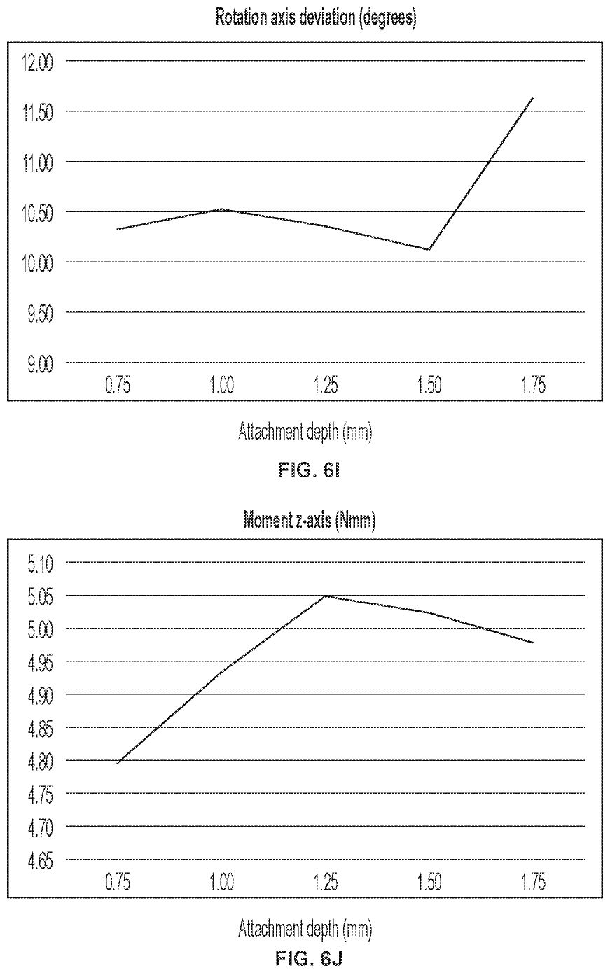

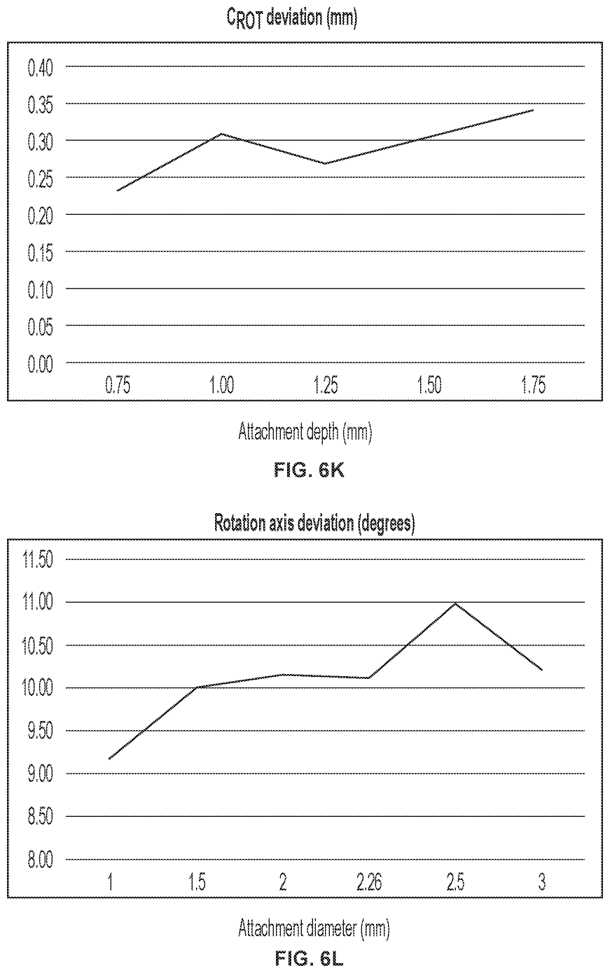

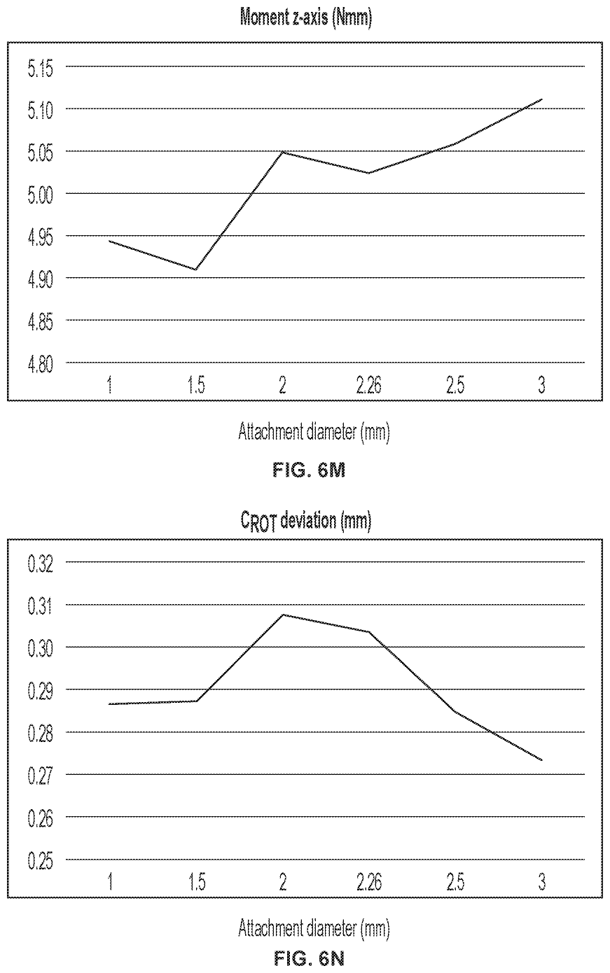

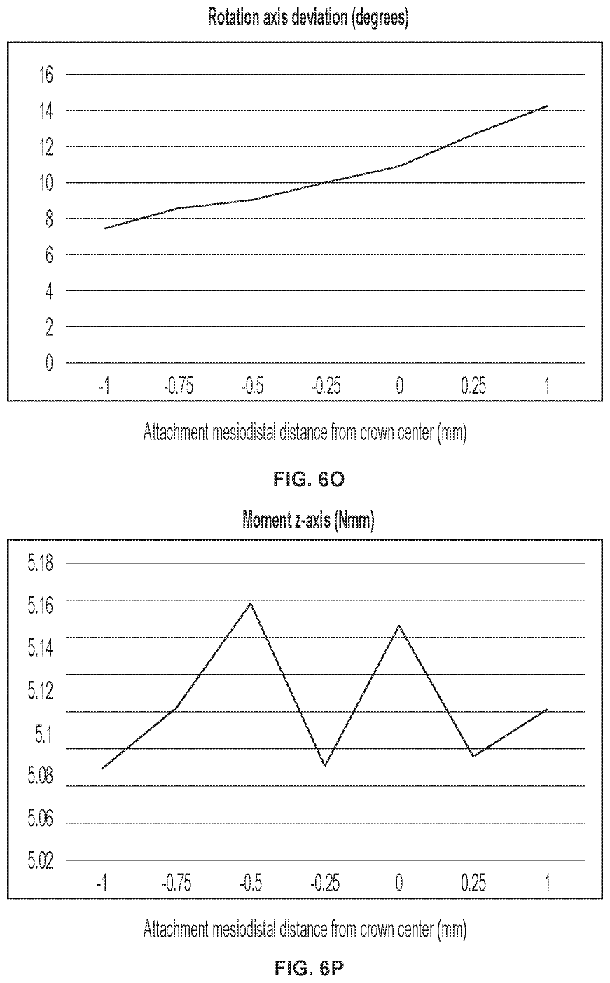

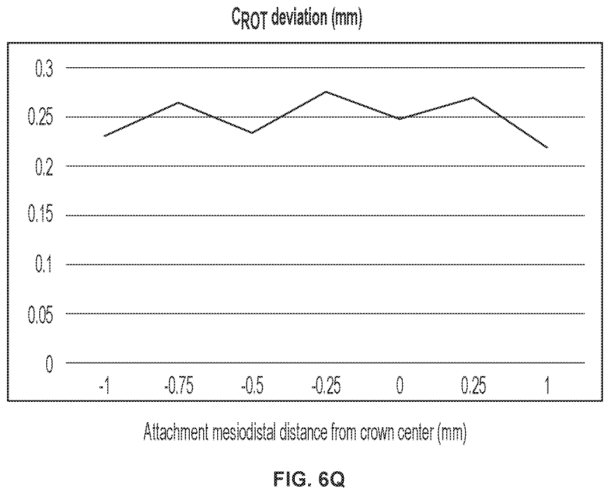

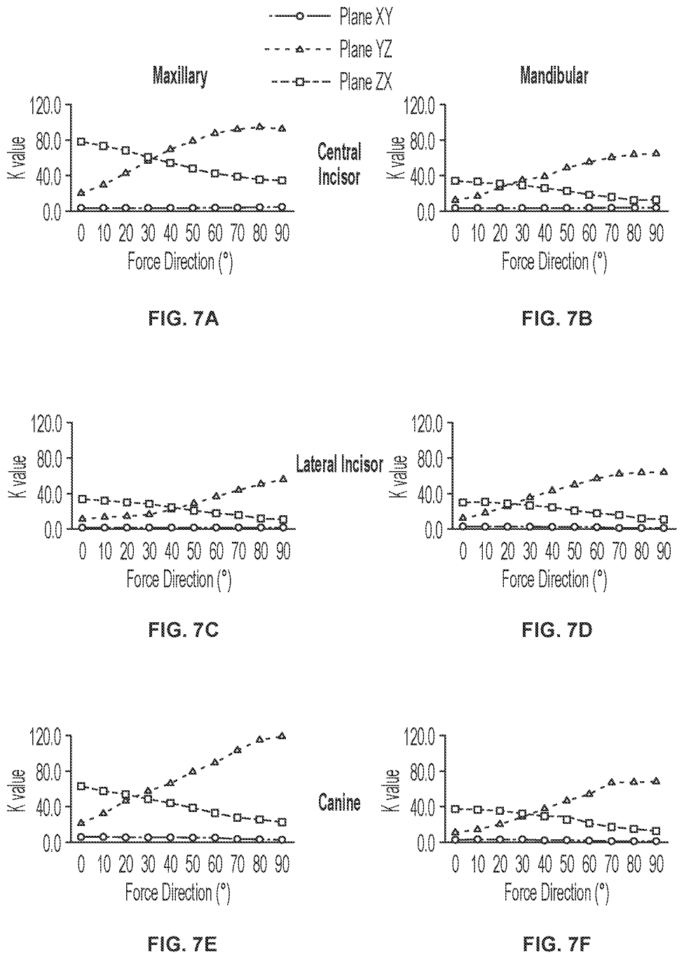

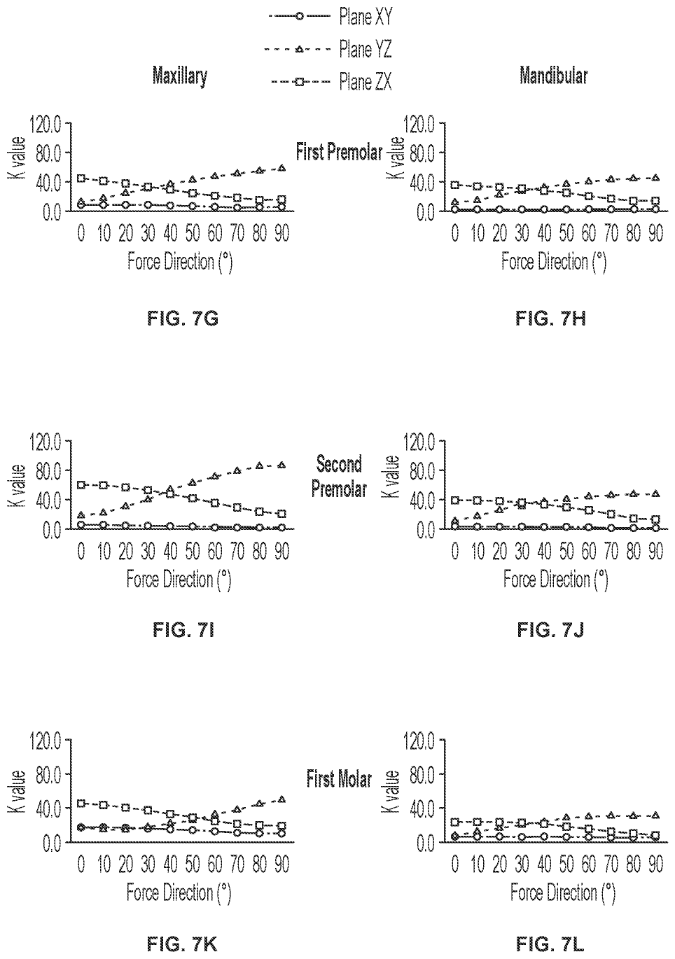

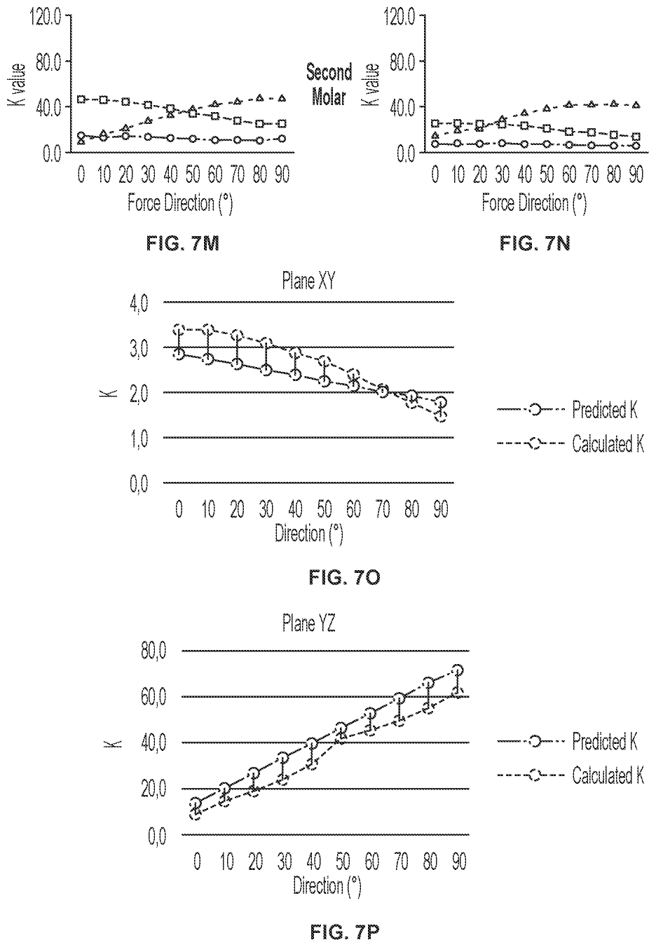

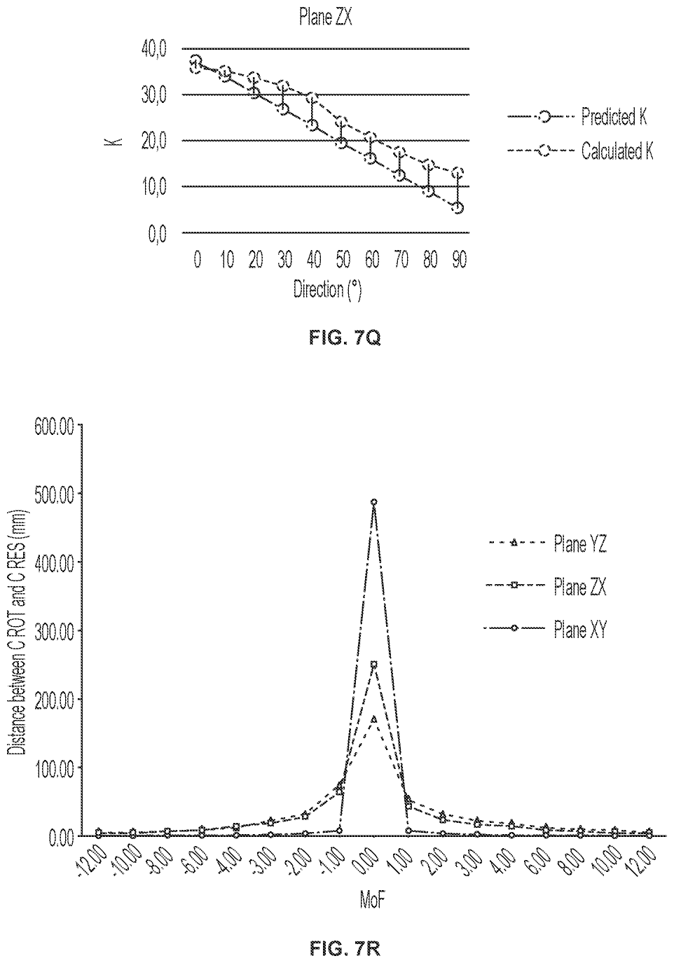

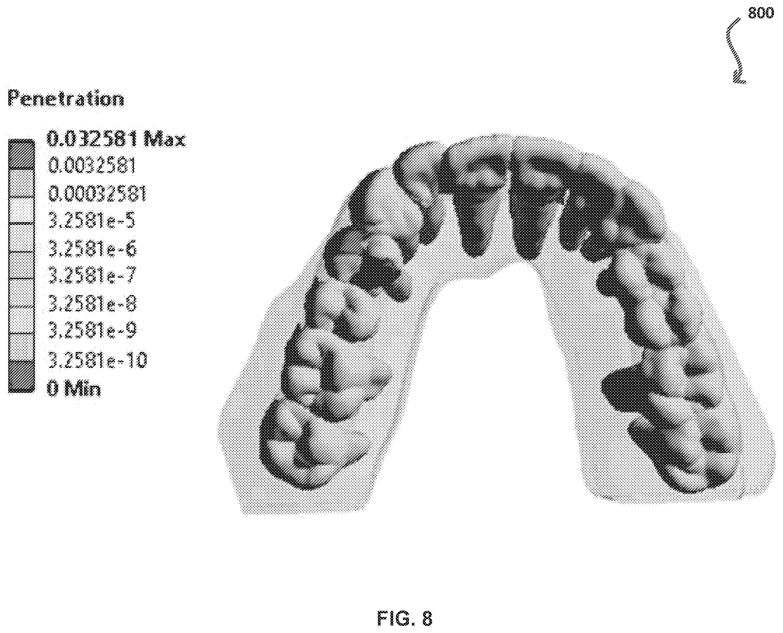

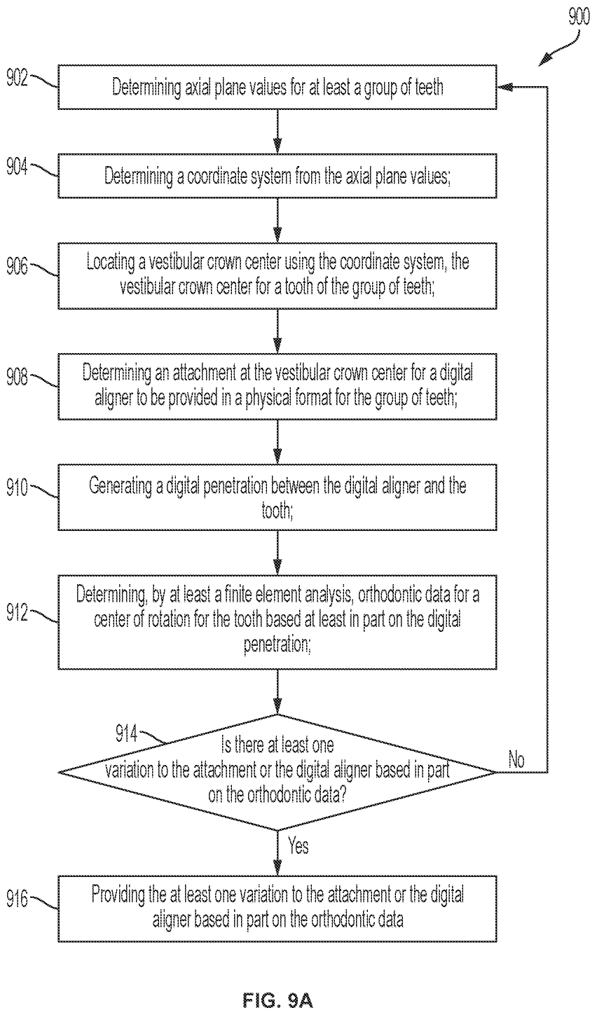

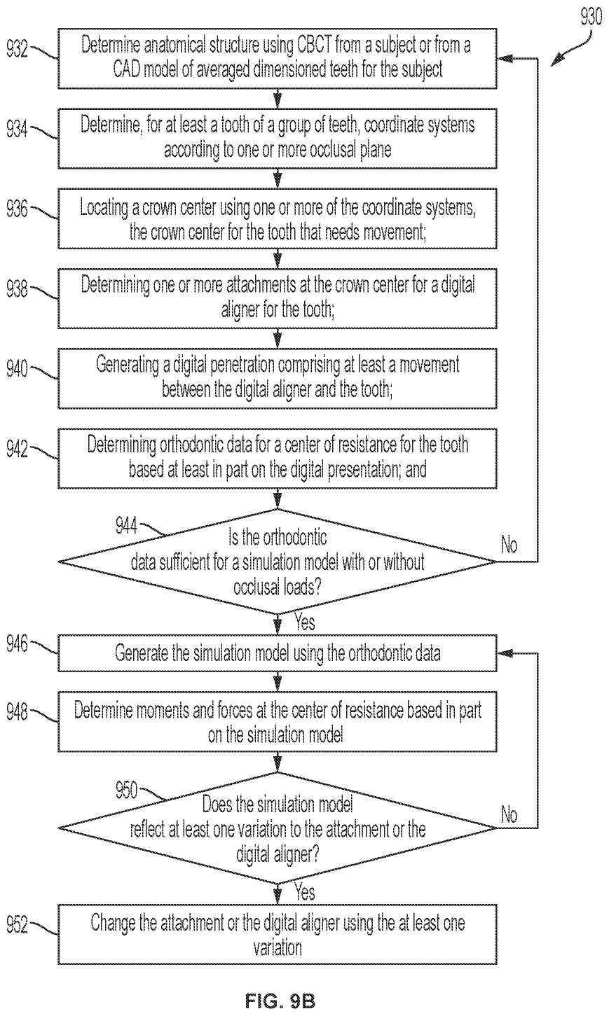

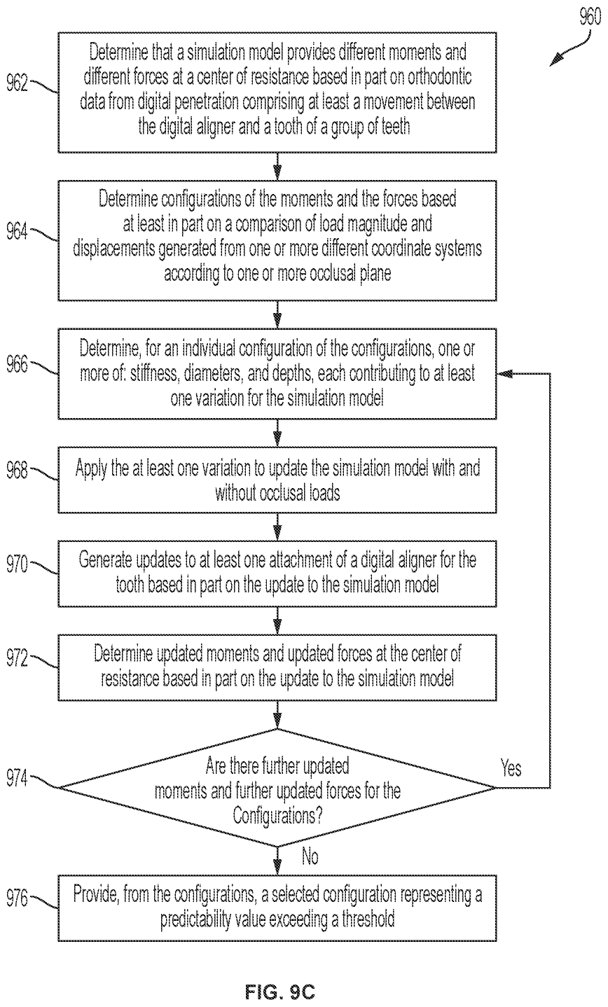

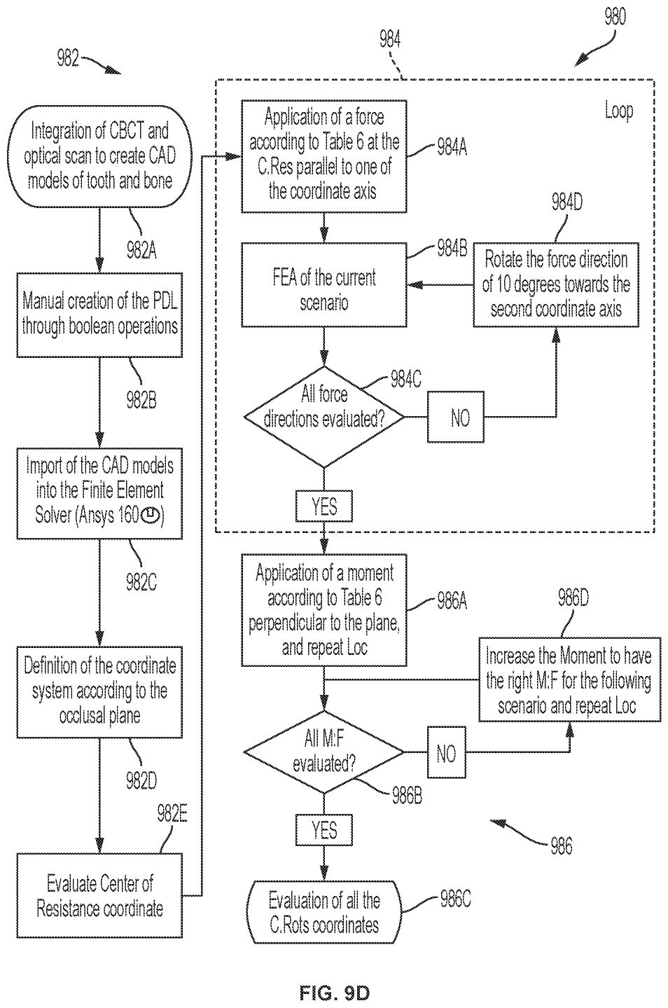

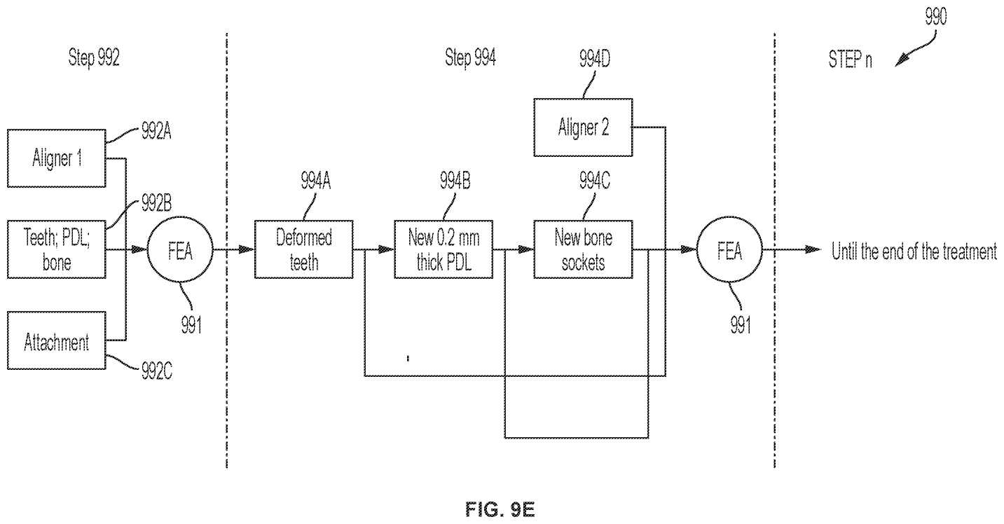

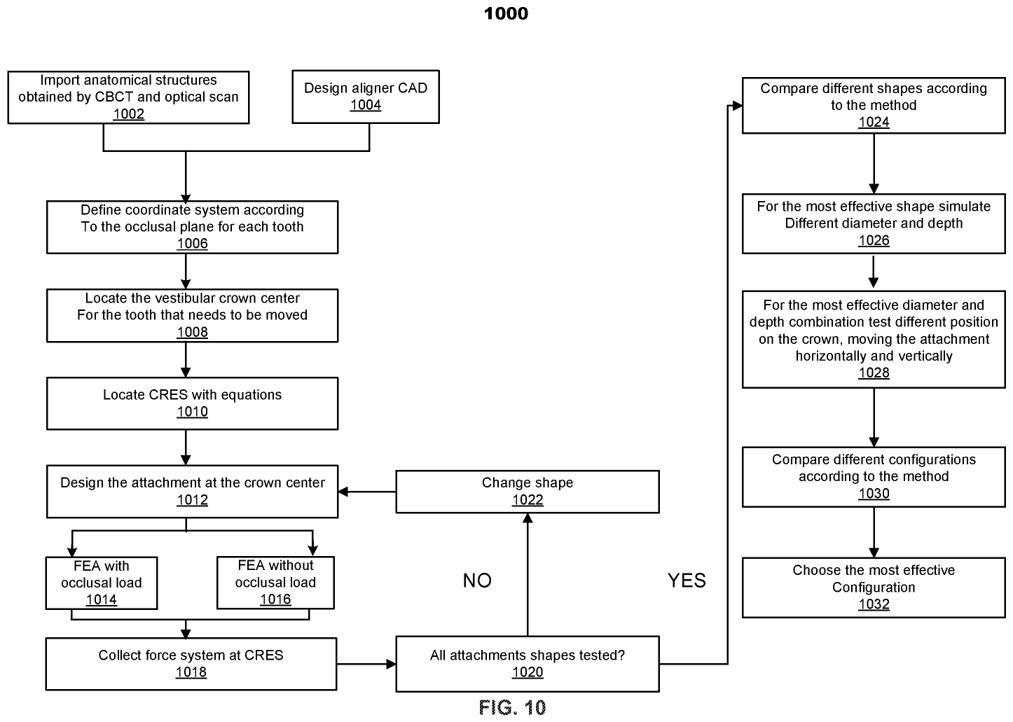

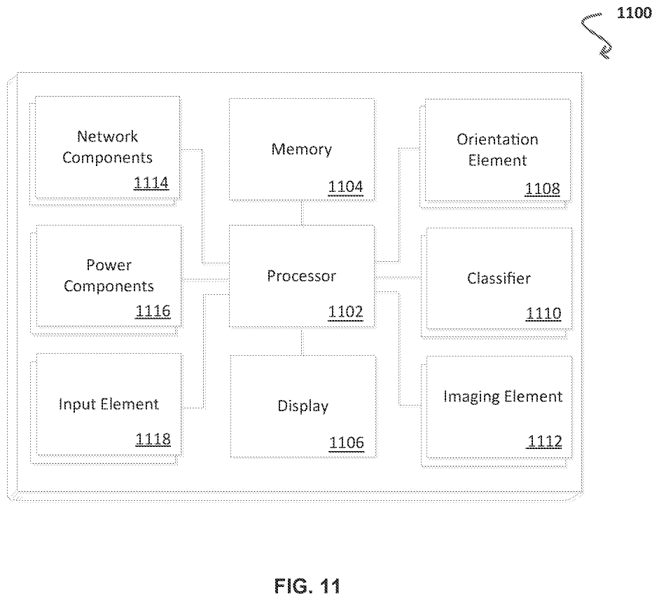

FIGS. 6L, 6M, and 6N are graphical representations of the deviation of the rotation axis, moment Z-axis, and the C_{ROT} deviation for different diameter of attachments and aligners in accordance with this disclosure.

FIGS. 60, 6P, and 6Q are graphical representations of the deviation of the rotation axis, moment Z-axis, and the C_{ROT} deviation for different mesiodistal distance from the crown center for an attachment in accordance with this disclosure.

FIGS. 7A and 7B are graphical representations of parameter k against values of force direction measured for maxillary and mandibular central incisors, respectively, on different planes, in an example from real patients.

FIGS. 7C and 7D are graphical representations of parameter k against values of force direction measured for maxillary and mandibular lateral incisors, respectively, on different planes, in an example from real patients.

FIGS. 7E and 7F are graphical representations of parameter k against values of force direction measured for maxillary and mandibular canines, respectively, on different planes, in an example from real patients.

FIGS. 7G and 7H are graphical representations of parameter k against values of force direction measured for maxillary and mandibular first premolars, respectively, on different planes, in an example from real patients.

FIGS. 7I and 7J are graphical representations of parameter k against values of force direction measured for maxillary and mandibular second premolars, respectively, on different planes, in an example from real patients.

FIGS. 7K and 7L are graphical representations of parameter k against values of force direction measured for maxillary and mandibular first molars, respectively, on different planes, in an example from real patients.

FIGS. 7M and 7N are graphical representations of parameter k against values of force direction measured for maxillary and mandibular second molars, respectively, on different planes, in an example from real patients.

FIGS. 70, 7P, and 7Q are graphical representations of the predicted and calculated values of the k parameter for simulation of attachments and aligners and for physical attachments and aligners in the XY plane, YZ plane, and ZX plane, respectively, in accordance with this disclosure.

FIG. 7R shows a graphical representation of a relationship between the moments and forces (M:F) and the distance between C_{ROT} and C_{RES} (“D_{C.Res-C.Rot}”) for an example tooth and an example angle in accordance with this disclosure.

FIG. 8 is an illustration of the penetration values obtained from movement, in the example simulation, between attachments and aligners and a tooth, along with adjacent teeth, in accordance with this disclosure.

FIGS. 9A, 9B, 9C, 9D, and 9E are flow diagrams of methods to perform the simulations of attachments and aligners in accordance with this disclosure.

FIG. 10 is a flow diagram of a method to perform the simulations of attachments and aligners in accordance with this disclosure.

FIG. 11 is a block diagram of a system with example components that can be used to implement aspects of various embodiments.

FIG. 12 illustrates an example system environment that can be used to implement aspects of various embodiments.

### DETAILED DESCRIPTION

So that the manner in which the features and advantages of the embodiments of systems for simulation of dental attachments or aligners and associated methods, as well as others, which will become apparent, may be understood in more detail, a more particular description of the embodiments of the present disclosure briefly summarized previously may be had by reference to the embodiments thereof, which are illustrated in the appended drawings, which form a part of this specification. It is to be noted, however, that the drawings illustrate only various embodiments of the disclosure and are therefore not to be considered limiting of the present disclosure's scope, as it may include other effective embodiments as well.

The method and system of the present disclosure will now be described more fully hereinafter with reference to the accompanying drawings in which embodiments are shown. The method and system of the present disclosure may be in many different forms and should not be construed as limited to the illustrated embodiments set forth herein; rather, these embodiments are provided so that this disclosure will be thorough and complete, and will fully convey its scope to those skilled in the art. Like numbers refer to like elements throughout. In an embodiment, usage of the term “about” includes +/−5% of the cited magnitude. In an embodiment, usage of the term “substantially” includes +/−5% of the cited magnitude.

It is to be further understood that the scope of the present disclosure is not limited to the exact details of construction, operation, exact materials, or embodiments shown and described, as modifications and equivalents will be apparent to one skilled in the art. In the drawings and specification, there have been disclosed illustrative embodiments and, although specific terms are employed, they are used in a descriptive sense only and not for the purpose of limitation.

In the following description, various embodiments will be described. For purposes of explanation, specific configurations and details are set forth in order to provide a thorough understanding of the embodiments and these embodiments may be used in any combination as may be readily understood by a person of ordinary skill upon reading the present disclosure. However, it will also be apparent to one skilled in the art that the embodiments may be practiced without the specific details. Furthermore, well-known features may be omitted or simplified in order not to obscure the embodiment being described.

Methods and systems presently disclosed addresses the aforementioned failures by providing digital simulations of an aligner and attachment set up for movements of individual tooth and/or groups of teeth. As such, the simulations represent options for selecting one of the aligner and/or attachment approach to resolve periodontal ligament deformations and take such issues into account during an initial visit to set the aligner into place. As the present process incorporates at least one variation in an iterative process to improve the simulation, the present process provides an optimal selection of the aligner and/or the attachment. This also represents a decrease in the number of visits required and the number of subsequent adjustments required to enable a level of comfort and a proper correction to the intended positions of the tooth and/or groups of teeth. Aligners may include attachments, and a person of ordinary skill, upon reading the present disclosure, would recognize that selection of an attachment corresponds to selection of an aligner to support the attachment. As such, this disclosure may refer to the selection of an attachment alone, but such a selection may then be extended to the aligner by selection of material and shapes that correspond between the attachment and aligner. Further, in view of the above, the discussion herein uses attachments and aligners interchangeably unless otherwise explicitly noted and a person of ordinary skill would understand from this discussion as to transferring requirements from an attachment to an aligner or an aligner to an attachment, and to the use of both as required.

The outcome of the method and system supports a new biomechanically centered aligner and attachment system that optimizes many features required to enable the aforementioned level of comfort and proper correction to the intended positions to secure a best shape for an aligner, attachment or group of attachments. For example, a simulation providing a selection of a biomechanically centered aligner and attachment system may then be used to generate an aligner and/or attachment system in a physical format by any available machining system, including by use of 3-dimensional (3D) printing or computer numeric control (CNC) machines. The features referenced above may include a first feature enabled by the present disclosure—of a system requiring a higher magnitude of force to move a tooth in a desired direction with a least magnitude of force directed to undesired directions of movement. This feature may be a quantitative criterion defined by threshold limitations applied in the simulation model to restrict the simulation model. A second feature enabled by the present disclosure is a feature of delivering a smallest distance between an intended center of rotation and an obtained center of rotation in the plane of movement. As with the prior feature, this may also be a qualitative criterion controlled in the simulation model as a variation applied in the simulation model, and which may then be applied in the physical format. Certain embodiments include an additional feature of delivering a smallest distance between an intended axis of rotation and an obtained axis of rotation in the plane of movement

A third feature enabled by the present disclosure is a feature of allowing a determined level of grip (geometric fit) of the aligner to the tooth or group of teeth, and in the direction of the movement that is desired for achieving control of the movement. As such, the level of grip achieved is generally the best in relation to prior trial-and-error methods. In an example, if a tooth needs to be rotated around a long axis of a prism-type attachment, then the simulation model and subsequent orthodontic data would support that the prism-type attachment be cut into a triangular shape. This shape would allow the attachment to perform better than other available shapes, such as a square shape or any subsequent multi-faceted prism shape, including a cylindrical shape.

A fourth feature enabled by the present disclosure is a feature of allowing a determined freedom for a geometric fit of same entities in multiple planes of space. For example, when a tooth is not required to be moved in a certain direction, then the simulation model and subsequent orthodontic data would reflect a cylindrical shape as being appropriate for the application in question. While these features may be performed independently, the present disclosure also supports a fifth feature that is a combination of two or more of the aforementioned features that are achieved by the simulation model, the orthodontic data, and any variations applied to the simulation model to update the simulation model. For example, the first two aforementioned features may be combined to achieve an equilibrium environment in terms of forces between the target tooth and adjacent teeth. In particular, the equilibrium environment may be as to anchorage teeth that support movement of the target tooth, and optionally of the adjacent teeth, with the forces being applied to achieve the equilibrium environment.

A sixth feature in the present disclosure supports differentially altering a stiffness of an aligner material in the simulation model, which will then provide for an aligner in the physical format that uses a wire or material with a larger elastic modulus, for example. Such an implementation accommodates the other features, including a higher magnitude of force system in an intended direction with least forces in unintended directions, the least distance of movement, and the determined level of grip to force the movement.

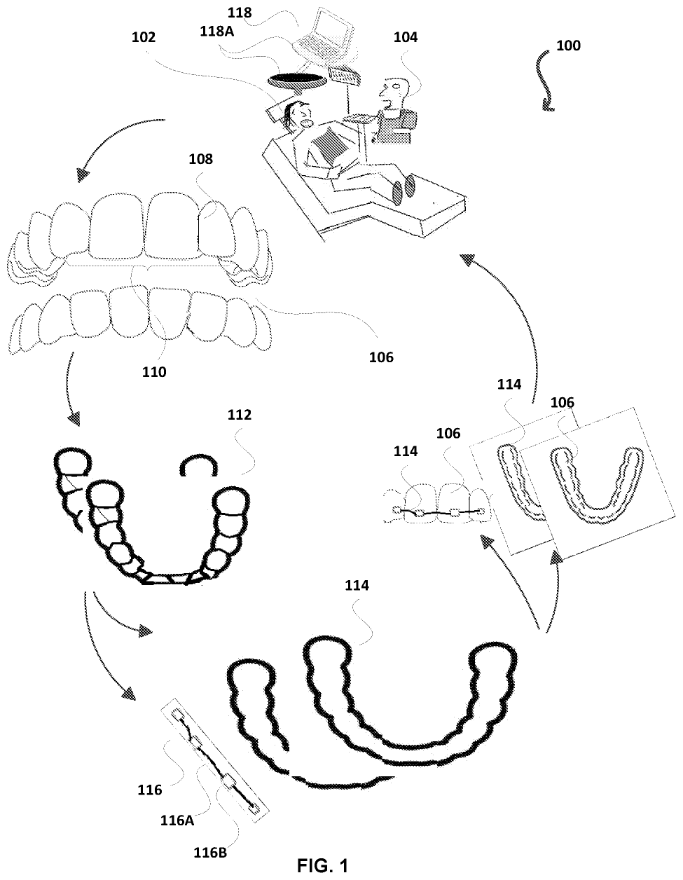

FIG. 1 is an example process flow 100 in accordance with an aspect of the present disclosure. Subject 102 is examined by a qualified professional 104 in facility 118 providing equipment 118A for orthodontic examination. Such equipment 118A may include one or more of, without limitation, a panorex imaging device, an x-ray imaging device, a cephalometric extension x-ray imaging device, a cone beam (CBCT) imaging device, camera imaging devices (e.g., intraoral cameras), periapical x-ray imaging device, an optical scanner, and/or a digital computer-aided design and manufacturing (CAD/CAM) machine. A target tooth 108 or group of teeth 110 from the general set of the subject's teeth 106 may be selected by the qualified professional 104 for assessment to adjust, reposition, or move into a desired position. One or more images 112 are captured of at least the target tooth 108 and/or the group of teeth 108, and may also be of the general set of the subject's teeth 106. The image may be analyzed by the qualified professional 104 with or without assistance from software on one or more of the equipment 118A. The qualified professional 104 works with a provider of the software and of the equipment 118A to provide attachments and/or aligners 114, 116 to be used with the subject's teeth 106. The attachments and/or aligners 114, 116 are particularly the focus on the provided attachments and/or aligners 114, 116. However, the subject 102 is required to revisit the facility 118 to ensure that any adjustment, repositioning, or movement of the tooth 108 and/or of the group of teeth 108 is as to the desired position. Such a verification and further adjustment is via the transducer approach, which tends to be rigid and somewhat misleading as noted elsewhere in this disclosure.

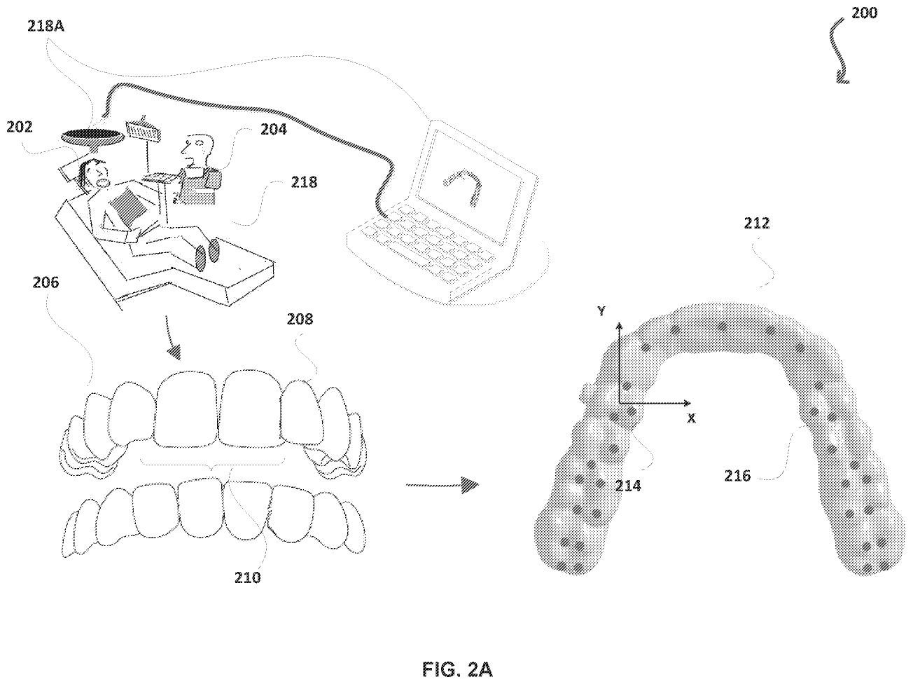

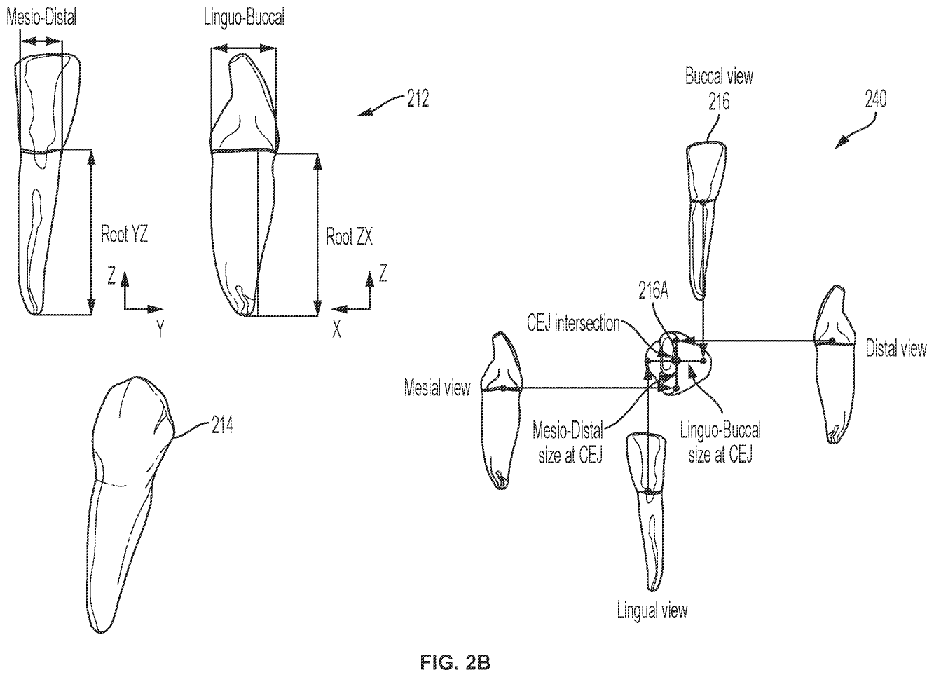

FIG. 2A is one aspect 200 of an example process flow for performing the present simulation of attachments and aligners in accordance with this disclosure. Subject 202 is examined by a qualified professional 204 in facility 218 providing equipment 218A for orthodontic examination. Such equipment 218A may include one or more of the equipment discussed with respect to FIG. 1. A target tooth 208 or group of teeth 210 from the general set of the subject's teeth 206 may be selected by the qualified professional 204 for assessment to adjust, reposition, or move into a desired position. One or more images 212 are captured of at least the target tooth 208 and/or the group of teeth 210, and may also be of the general set of the subject's teeth 206. The image may be analyzed by the qualified professional 204 with or without assistance from software on one or more of the equipment 118A. The equipment 118A may additionally include software and features are presently disclosed for using simulation models to predict teeth movement and to propose placement of the attachment and/or aligner in a digital manner. This digital manner may be by way of illustrative orthodontic data or by numeric-based orthodontic data underlying the illustrative orthodontic data. In this disclosure the illustrative or numeric-based orthodontic data is used interchangeably with orthodontic data to refer to any data realized from the simulation models and subsequent processes.

The qualified professional 204 works with a provider of the software and of the equipment 218A to provide physical format attachments and/or aligners to be used with the subject's teeth 206 from the simulated or digital attachments and/or aligners. The physical format attachments and/or aligners generated using the simulation model and subsequent processes are long-term attachments and/or aligners that take into consideration most changes the subject 202 may undergo due to natural growth and changes from the initial positions proposed for the physical format attachments and/or aligners. As such, the subject 202 may not be required to revisit the facility 218. One aspect of the present simulation provides two options for each scenario to evaluate the threshold boundary conditions during treatment. In a first simulation, there is no accountability in place based in part on restrictions for displacement of a digital aligner (and subsequently, for the physical format aligner). This simulation may correspond to a condition representing phases or future changes when there is no contact between a mandibular and a maxillary arch and when the mouth is slightly open. In a second simulation, there is no accountability in place based in part on restrictions for the displacement of the digital aligner (and subsequently, for the physical format aligner) in the direction perpendicular 214 to the occlusal plane 216. The occlusal points on the external aligner surface are restricted from any movement. Occlusal plane, along with other axis used to determine movement of the teeth are referred to herein as axial planes. The simulation, as prepared above, may correspond to a completely closed mouth of subject 202. As such, a physical format aligner based on such simulations accounts for an occlusal pressure on an area of contact with the opposite arch that cannot have vertical displacement.

FIG. 2B illustrates example dental features 240 observed or determined for the present simulation of attachments and aligners in accordance with this disclosure. The dental figures 240 are provided in the graphical representations 212, 214, and 216 of the teeth dimensions used to predict certain features used in the simulation models. Different views are illustrated in FIG. 2B to visualize an application of force and moment at different positions (or views or dimensions) at issue to achieve different movements for different tooth types and/or groups of teeth.

The present disclosure clarifies a nonlinear relationship that may exist between tooth movement and force system directions. In an example, a formula known as the Burstone formula may need modification to incorporate the tooth movement and force system directions presently disclosed. A person of ordinary skill, with the knowledge of the Burstone formula will readily understand the required modifications on reading the presently disclosed methods and system. Given two force systems, system A and system B, in different directions, and given two displacements A′ and B′ of a tooth or group of teeth, an average vectorial force system (A+B)/2 may not necessarily result in an average displacement (A′+B′)/2. As such, different types of nonlinear behavior depend on the applied force and moment directions.

Systems and methods disclosed here take into consideration and root asymmetries, as well as specific differences in tooth morphology are presently incorporated into the simulation models to resolve this issue. As a result, tooth morphology as provided by morphological characteristics of the tooth is relied upon to determine this nonlinear behavior. Statistical analysis is a basis to determine tooth movement in any direction as a prediction when data on root morphology and the original force system are provided to or obtained by the professional as noted with regard to FIG. 2A. Basic tooth morphology parameters are assumed and tested, statistically, to provide mathematical laws governing tooth movement for a tooth and/or of groups of teeth, where such movement are represented using linear root dimensions for the tooth on the three main coordinate axes, the number of roots, and the crown length.

Dental features 212 illustrate axes for Mesio-Distal and for Linguo-Buccal dimensions, while dental features 214 provides a simplistic coordinate axes for a maxillary canine, and dental features 216 provides a coordinate system for a cement-enamel-junction (CEJ). Particularly, the CEJ intersection is illustrated at the center 216A of the illustrated tooth. The CEJ intersection may refer to an intersection of the center of resistance (C_{RES}) coordinates defined on the plane XY by the intersection between the Mesio-Distal and the Linguo-Buccal tooth dimensions. On the Z-axis, a coordinate system is located at the average between CEJ on the Mesio-Distal dimensions and CEJ on the Linguo-Buccal dimensions. In an example, penetration data is generated for each simulation using moment and forces (M/F or M:F) values at the C_{RES} of the tooth and using distances between the intended and obtained centers of rotation. Here, to perform the above determination, the morphology of the teeth is reflected in a parameter k, which depends on tooth dimensions and force system features. A k parameter dataset is generated for a subject's tooth using parameters—e.g., the axes values recited above—or is generated for a general or random tooth using a CBCT analysis and using a specific set of predictors as described throughout this disclosure. All dimensions disclosed herein may be measured at the tooth CEJ and the two axes intersecting at center 216A. The average root length may be calculated as the average between Root ZX and root YZ axes in dental features 212. The above processes provide a determination of axial plane values for at least a group of teeth and also support determination of the coordinate system from the axial plane values. The center of resistance generally depends on tooth morphology and remains constant, and thus, the center of resistance is typically measured prior to starting the attachment simulation, e.g., through multiple, such as 3, FEA simulations, although alternatively it is possible to calculate its approximate coordinates using the predictors from Tables 8 and 9. Table 9 lists the predictors to calculate an approximate Center of resistance location. Table 8 lists the Center of resistance locations measured using FEA for a set of teeth.

A set of predictors can be applied to a generic tooth when the C_{RES} coordinates are known. The above-referenced Burstone formula may be used to provide a relationship between tooth dimensions and a C_{RES} location on the tooth along an axis for the tooth, for example. In the present disclosure, a statistically based 3D mathematical relationship is obtained for C_{RES} and the tooth morphology. In an example, the C_{RES} coordinates for a known dataset of a general set of teeth is used to introduce a set of predictors. From the predictors, the 3D C_{RES} coordinates are determined based on the tooth dimensions. In another example, the predictors may be obtained by statistical analysis on the C_{RES} locations of 14 maxillary and 14 mandibular teeth, so the power and accuracy is naturally limited by this sample size. This may be based in part on a set of teeth from a subject wishing to receive treatment for repositioning, adjusting, or correcting teeth positions.

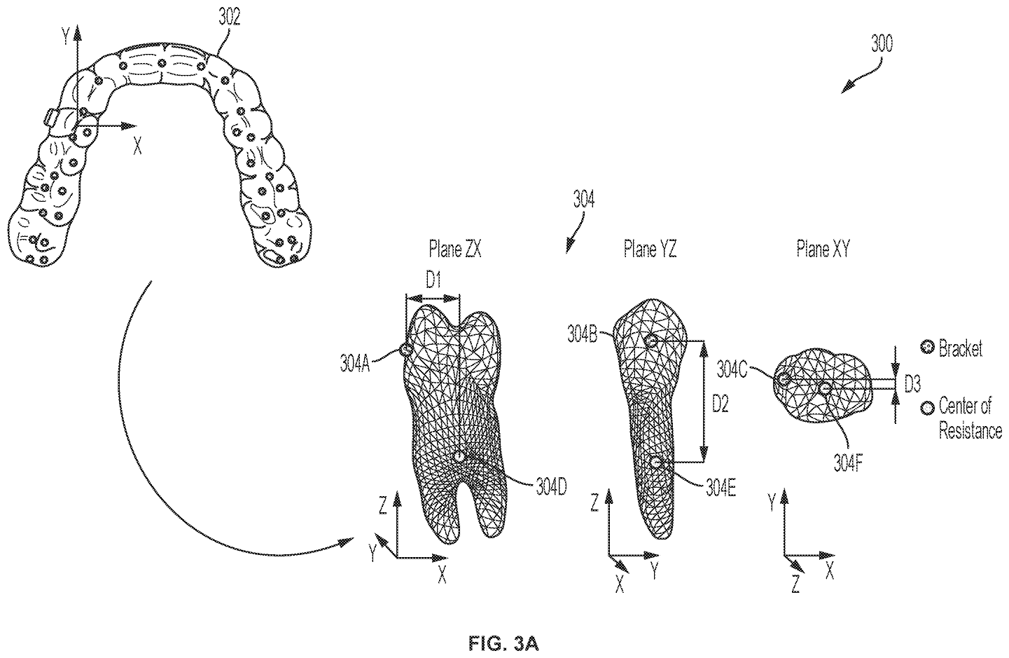

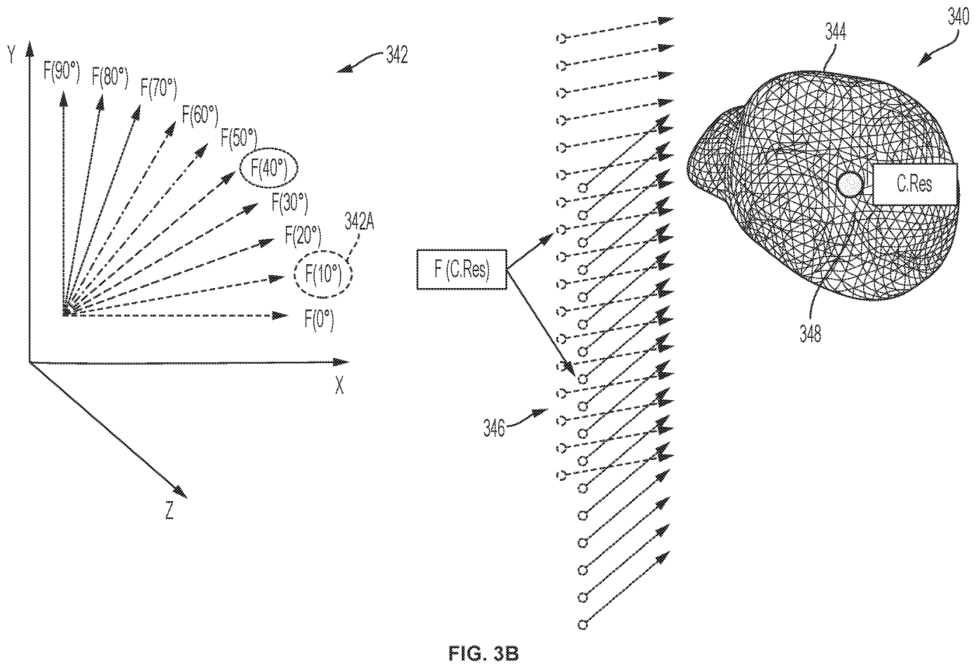

FIG. 3A is another aspect 300 of an example process flow for performing the present simulation of attachments and aligners in accordance with this disclosure. Particularly, FIG. 3A illustrates the generation or determination of k values in PLANE XY, PLANE YZ, and PLANE ZX, which provide the morphology of the teeth 302. These values may be determined using the previously discussed statistical methods for a general set of teeth or for a set of teeth from a subject and by incorporating predictor values to generate the C_{RES} coordinates.

In one embodiment, k values can be calculated or generated based on one or more predictors/coefficients, such as shown in TABLE 1. In TABLE 1, k coefficients/predictors for each tooth are shown, and LB is related to the linguobuccal dimension at CEJ; MD is related to the mesiodistal dimension at CEJ; R_{YZ} is related to the root length on the plane YZ; R_{ZX} is related to the root length on the plane ZX; and R_{AVG} is related to the average between R_{YZ} and R_{ZX}. The predictors used to calculate “k” can include the unstandardized coefficients, though standardized coefficients can be used without departing from the scope of the present disclosure. In some variations, the parameter k can be predicted with the set of k predictors/coefficients shown in TABLE 1 at a statistically significant level (P<0.05). It further is possible to retrieve different equations for each tooth and planes and thus “k” can be estimated for every tooth in any direction in this manner, for example, k for tooth LR1 and plane XY, can be calculated as k=−2.925+0.436*R_{YZ}-0.012*Angle.

TABLE 1

| | | | Unstandardized | | | 95.0% Confidence | |
|---|---|---|---|---|---|---|---|
| | | | Coefficients | | | Interval for B | |
| | | | | Std. | | Lower | Upper |
| Tooth | Plane | Model | B | Error | Sig. | Bound | Bound |
| LR1 | XY | (Constant) | −2.925 | .408 | .000 | −3.799 | −2.051 |
| | | R_{YZ} (mm) | .436 | .033 | .000 | .366 | .506 |
| | | Angle [°] | −.012 | .001 | .000 | −.014 | −.010 |
| | YZ | (Constant) | 70.928 | 17.556 | .002 | 32.288 | 109.568 |
| | | Angle [°] | .642 | .043 | .000 | .547 | .738 |
| | | R_{AVG}(mm) | −4.224 | 1.364 | .010 | −7.226 | −1.221 |
| | ZX | (Constant) | 94.826 | 12.679 | .000 | 67.200 | 122.452 |
| | | R_{YZ} (mm) | −4.309 | 1.011 | 001 | −6.511 | −2.107 |
| | | Angle [°] | −.353 | .030 | .000 | −.418 | −.288 |
| LR2 | XY | (Constant) | −2.726 | .860 | .009 | −4.619 | −.834 |
| | | Angle [°] | −.018 | .002 | .000 | −.022 | −.013 |
| | | R_{ZX} (mm) | .453 | .072 | .000 | .294 | .613 |
| | YZ | (Constant) | 11.913 | 2.969 | .003 | 5.196 | 18.629 |
| | | Angle [°] | .641 | .052 | .000 | .524 | .758 |
| | ZX | (Constant) | 1.727 | 7.641 | .827 | −15.892 | 19346 |
| | | Angle [°] | −.269 | .015 | .000 | −.303 | −.236 |
| | | R_{ZX} (mm) | 2.457 | .608 | .004 | 1.056 | 3.858 |
| LR3 | XY | (Constant) | −.172 | .363 | .651 | −1.031 | .688 |
| | | Angle [°] | −.017 | .001 | .000 | −.019 | −.015 |
| | | R_{AVG}(mm) | .240 | .028 | .000 | .173 | .307 |
| | YZ | (Constant) | 9.798 | 3.529 | .027 | 1.454 | 18.142 |
| | | Angle [°] | .707 | .077 | .000 | .525 | .890 |
| | ZX | (Constant) | −82.169 | 9.510 | .000 | −102.567 | −61.771 |
| | | Angle [°] | −.249 | .017 | .000 | −.286 | −.211 |
| | | LB (mm) | 20.289 | 1.692 | .000 | 16.660 | 23.919 |
| LR4 | XY | (Constant) | −.084 | .287 | .775 | −.723 | .554 |
| | | Angle [°] | −.007 | .00 | .000 | −.009 | −.006 |
| | | R_{AVG}(mm) | 193 | .027 | .000 | 132 | .253 |
| | YZ | (Constant) | −39.448 | 16.051 | .029 | −74.123 | −4.772 |
| | | Angle [°] | .370 | .025 | .000 | .316 | .423 |
| | | MD (mm) | 11.431 | 3.467 | .006 | 3.940 | 18.922 |
| | ZX | (Constant) | 35.153 | .945 | .000 | 32.975 | 37.331 |
| | | Angle [°] | −.257 | .018 | .000 | −.299 | −.216 |
| LR5 | XY | (Constant) | −3.723 | .891 | .002 | −5.708 | −1.737 |
| | | Angle [°] | −.013 | .002 | .000 | −.019 | −.008 |
| | | R_{ZX} (mm) | .466 | .065 | .000 | .321 | .610 |
| | YZ | (Constant) | 12.244 | 1.535 | .000 | 8.866 | 15.623 |
| | | Angle [°] | .502 | .03 | .000 | .434 | .569 |
| | ZX | (Constant) | 45.015 | 1.489 | .000 | 41.798 | 48.232 |
| | | Angle [°] | −.342 | .030 | .000 | −.406 | −.278 |
| LR6 | XY | (Constant) | −3.485 | 1.145 | 014 | −6.076 | −.893 |
| | | Angle [°] | −.009 | .003 | .009 | −.015 | −.003 |
| | | LB (mm) | .988 | 128 | .000 | .697 | 1.278 |
| | YZ | (Constant) | −30.629 | 8.343 | .004 | −48.991 | −12.267 |
| | | Angle [°] | .258 | .019 | .000 | 216 | .300 |
| | | LB (mm) | 4.484 | .920 | 000 | 2.459 | 6.509 |
| | ZX | (Constant) | 25.996 | 1.036 | .000 | 23.653 | 28.339 |
| | | Angle [°] | −.210 | .017 | .000 | −.249 | −.172 |
| LR7 | XY | (Constant) | −17.535 | .804 | .000 | −19.435 | −15.634 |
| | | Angle [°] | −.018 | .002 | .000 | −.023 | −.013 |
| | | R_{AVG}(mm) | 2.070 | .071 | .000 | 1.902 | 2.237 |
| | YZ | (Constant) | 21.989 | 2.171 | .000 | 16.981 | 26.996 |
| | | Angle [°] | 221 | .045 | .001 | 117 | .325 |
| | ZX | (Constant) | −5.848 | 3.328 | 102 | −13.038 | 1.341 |
| | | R_{YZ} (mm) | 2.834 | .304 | 000 | 2.176 | 3.491 |
| | | Angle [°] | −.147 | .008 | .000 | −.165 | −.130 |
| UR1 | XY | (Constant) | 2.404 | 4.532 | .000 | 1.938 | 2.869 |
| | | Angle [°] | −0.003 | .082 | −0.483 | −0.012 | 0.006 |
| | YZ | (Constant) | 125.133 | 34.322 | .007 | 45.987 | 204.280 |
| | | Angle [°] | .910 | .068 | 000 | 754 | 1.065 |
| | | MD (mm) | −16.136 | 5.614 | .021 | −29.082 | −3.190 |
| | ZX | (Constant) | 191.353 | 16.088 | .000 | 156.300 | 226.407 |
| | | Angle [°] | −.505 | .032 | .000 | −.575 | −.434 |
| | | MD (mm) | −18.964 | 2.437 | .000 | −24.273 | −13.656 |
| UR2 | XY | (Constant) | 11.043 | .676 | .000 | 9.485 | 12.601 |
| | | Angle [°] | −.012 | .001 | .000 | −.014 | −.009 |
| | | LB (mm) | −1.389 | .111 | .000 | −1.646 | −1.132 |
| | YZ | (Constant) | 68.928 | 14.388 | .000 | 37.845 | 100.012 |
| | | Angle [°] | .490 | .030 | .000 | .427 | .554 |
| | | R_{ZX} (mm) | −3.791 | .991 | .002 | −5.932 | −1.650 |
| | ZX | (Constant) | −75.939 | 23.618 | .011 | −129.368 | −22.511 |
| | | Angle [°] | −.330 | .039 | .000 | −.419 | −.241 |
| | | LB (mm) | 19.624 | 3.893 | .001 | 10.818 | 28.430 |
| UR3 | XY | (Constant) | −1.744 | .707 | .027 | −3.260 | −.228 |
| | | Angle [°] | −.031 | .002 | .000 | −.035 | −.027 |
| | | LB (mm) | .904 | .087 | .000 | .717 | 1.091 |
| | YZ | (Constant) | 28.181 | 4.599 | .000 | 17.933 | 38.429 |
| | | Angle [°] | 1.074 | .085 | .000 | .885 | 1.262 |
| | ZX | (Constant) | 61.654 | 2.497 | 000 | 56.214 | 67.094 |
| | | Angle [°] | −.492 | .044 | .000 | −.588 | −.396 |
| UR4 | XY | (Constant) | 28.903 | 2.270 | .000 | 23.958 | 33.848 |
| | | Angle [°] | −.037 | .005 | .000 | −.048 | −.026 |
| | | MD (mm) | −4.123 | .438 | .000 | −5.077 | −3.170 |
| | YZ | (Constant) | −46.292 | 19.376 | .044 | −90.973 | −1.610 |
| | | Angle [°] | .578 | .013 | .000 | .548 | .608 |
| | | LB (mm) | 6.891 | 2.258 | .016 | 1.685 | 12.098 |
| | ZX | (Constant) | 68.744 | 6.541 | .000 | 54.613 | 82.875 |
| | | Angle [°] | −.321 | .012 | .000 | −.347 | −.295 |
| | | MD (mm) | −5.287 | 1.256 | .001 | −8.000 | −2.574 |
| UR5 | XY | (Constant) | −25.264 | 2.936 | .000 | −31.561 | −18.967 |
| | | Angle [°] | −.034 | .003 | .000 | −.041 | −.027 |
| | | R_{AVG}(mm) | 2.182 | .210 | .000 | 1.733 | 2.632 |
| | YZ | (Constant) | 20.307 | 1.850 | .000 | 16.277 | 24.337 |
| | | Angle [°] | .810 | 034 | .000 | .735 | .885 |
| | ZX | (Constant) | 65.308 | 1.094 | .000 | 62.962 | 67.654 |
| | | Angle [°] | −.467 | .020 | .000 | −.509 | −.424 |
| UR6 | XY | (Constant) | 18.281 | .629 | .000 | 16.896 | 19.666 |
| | | Angle [°] | −.116 | .011 | .000 | −.140 | −.092 |
| | YZ | (Constant) | −20.170 | 14.287 | 188 | −52.002 | 11.663 |
| | | Angle [°] | .464 | .05 | .000 | .351 | 577 |
| | | MD (mm) | 3.969 | 1.766 | .048 | .034 | 7.905 |
| | ZX | (Constant) | 355.044 | 83.284 | .001 | 173.584 | 536.504 |
| | | Angle [°] | −.291 | .032 | .000 | −.360 | −.221 |
| | | LB (mm) | −34.624 | 9.363 | .003 | −55.026 | −14.223 |
| UR7 | XY | (Constant) | −6.120 | 4.054 | .157 | −14.954 | 2.713 |
| | | R_{YZ} (mm) | 1.676 | .316 | .000 | .989 | 2.364 |
| | | Angle [°] | −.072 | .004 | .000 | −.082 | −.062 |
| | YZ | (Constant) | 161.592 | 27.514 | .000 | 99.350 | 223.833 |
| | | Angle [°] | .346 | .034 | .000 | .268 | .423 |
| | | LB (mm) | −14.270 | 2.637 | 000 | −20.236 | −8.304 |
| | ZX | (Constant) | 193.416 | 17.450 | 000 | 155.396 | 231.436 |
| | | Angle [°] | −.240 | .018 | .000 | −.279 | −.201 |
| | | R_{AVG}(mm) | −12.016 | 1.386 | .000 | −15.036 | −8.996 |

In additional or alternative embodiments, the equations in TABLE 2 can be used to generate or to determine parameter k. The equations incorporate predictors for each tooth. The equations may change depending on the plane at issue and/or roots. In the equation, for example, X represents the tooth Linguo-Buccal length or size at the CEJ, Y represents the tooth Mesio-Distal length or size at the CEJ, Z represents a maximum Root length or size on the Linguo-Buccal direction, and A represents an angle between the force and an axis of the coordinate plane. The coordinate axis includes the Z-axis that is perpendicular to the occlusal plane, the Y-axis that is parallel to the occlusal plane and that follows the Mesio-Distal direction, and the X-axis that is parallel to the occlusal plane and follows the Linguo-Buccal direction.

TABLE 2

| | | |
|---|---|---|
| PLANE XY | 1 ROOT | k = −4.104 + 0.319Z − 0.534Y + 0.797X − 0.018A |
| | >1ROOT | k = −13.828 − 1.078Z − 1.405Y + 2.457X − 0.04A |
| PLANE YZ | 1 ROOT | k = −71.339 + 3.713Z + 3.960Y + 1.718X + 0.686A |
| | >1ROOT | k = −27.229 + 4.001Z + 0.871Y − 2.284X + 0.391A |
| PLANE ZX | 1 ROOT | k = −10.189 + 1.770Z + 8.497Y − 1.540X − 0.354A |
| | >1ROOT | k = −13.266 + 1.285Z − 1.444Y + 4.911X − 0.258A |

The numerically represented orthodontic data is provided in illustrative format via illustrations 304 for each PLANE value. Predicted or projected initial placements of attachments (e.g., brackets) 304A, 304B, and 304C, and their corresponding C_{RES} 304D, 304E, and 304F are also provided. The marked distance values D1, D2, and D3 are distances measured between each bracket and C_{RES} on each direction for a maxillary first premolar. For example, D1 is a distance function represented by Distance (C_{RES}-Bracket) on the Linguo-Buccal axis (X), D2 is a distance function represented by Distance (C_{RES}-Bracket) on direction perpendicular to Occlusal Plane (Z), and D3 is a distance function represented by Distance (C_{RES}-Bracket) on Mesio-Distal axis (Y).

With D1, D2, and D3 determined, Force Direction values are determined based in part on angles established by the axis in question, such as Linguo-Buccal, Occlusal Plane or Mesio-Distal axis. These values are provided in equations (1)-(3), along with the MF values at C_{RES} to provide the MF at Bracket points, MF_{Bracket}:

$$\begin{matrix}PLANE XY & \\ \frac{\text{?}}{\sqrt{1 + 1 / tan ( Force Direction )}} & Eq . ( 1 )\end{matrix}$$

$$\begin{matrix}PLANE YZ & \\ \frac{\text{?}}{\sqrt{1 + tan ( Force Direction )}} & Eq . ( 2 )\end{matrix}$$

$$\begin{matrix}PLANE ZX & \\ \frac{\text{?}}{\sqrt{1 + tan ( Force Direction )}} & Eq . ( 3 )\end{matrix}$$

$$\text{?} \text{indicates text missing or illegible when filed}$$

Where:
D1=Distance (C_{RES}-Bracket) on bucco-lingual direction (x)
D2=Distance (C_{RES}-Bracket) on direction perpendicular to Occlusal Plane (z)
D3=Distance (C_{RES}-Bracket) on mesio-distal direction (y)

Distance measurements D1, D2, and D3 are shown in FIG. 3A.

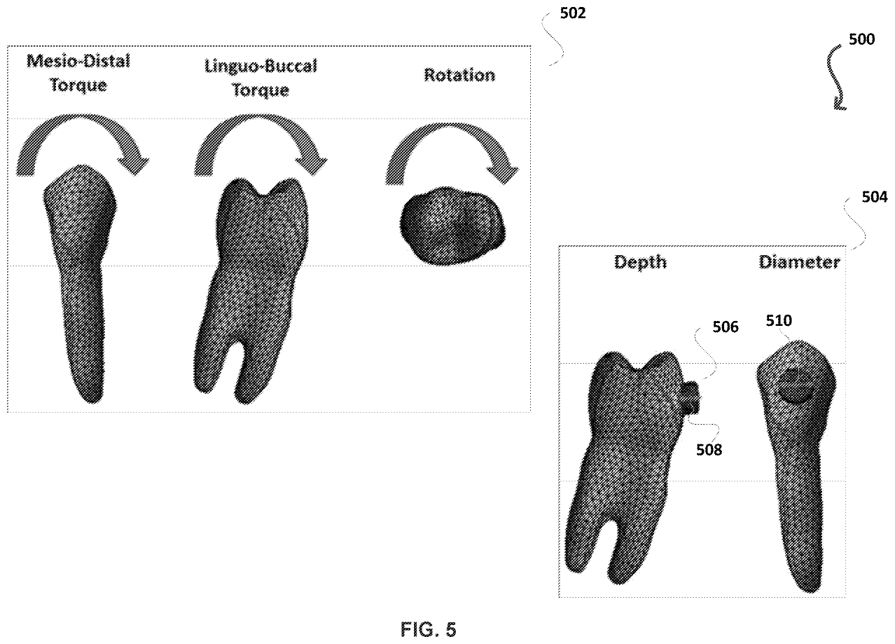

From the above discussion, it is apparent to a person or ordinary skill that the present disclosure determines the relationships between all meaningful permutations of M:F ratios and a projected or expected center of rotation (C_{ROT}), for different force directions, in the only available axial plane that supports rotation—as illustrated in FIG. 5. The projected or expected center of rotation (i.e., C_{ROT}) is another parameter that is then evaluated, in each scenario, through the displacement vectors of two nodes of the tooth. For each of the axial planes, multiple nodes may be selected. For example, two nodes may be selected such that they incorporate as much distance between them as possible in the axial plane. Such a process addresses issues where the tooth is not an ideal rigid body that may react in an expected manner to moments and forces. As such, a tooth may deform with higher relative error to the C_{ROT} location if the nodes are too close together. The center of rotation can describe/related to tooth movement and therefore is constantly changing, and it further is possible to calculate the center of rotation using “k” and knowing the resulting Force system delivered to the tooth by an orthodontic device.

The resulting C_{ROT} coordinates and the distances from the C_{RES} are evaluated and analyzed based in part on a mathematical relationship between M:F ratios and C_{ROT} as provided in equation (4). For example, taking Distance, D), as a difference between C_{RES} and C_{ROT}, and taking corresponding M:F ratios for distance D, parameter k establishes a relationship for M:F ratios and C_{ROT} for the target tooth, in its corresponding axial plane. This provides a location for a crown location, such as a crown center or other suitable location, using the coordinate system. The crown location, such as the crown center, is for a target tooth to be repositioned, adjusted, or moved, along with its adjacent teeth forming a group of teeth. Equation (4) is a simple hyperbolic equation taken to establish a relationship between C_{RES} and C_{ROT}.

$${D}_{C . Res - C . Rot} = \frac{k}{MF} \qquad Eq . ( 4 )$$

The knowledge of the relationship between C_{RES} and expected C_{ROT} helps properly position the bracket or attachment using the previously provided distance equation from the C_{RES} for D1, D2, and D3, and the predicted M:F ratios from equations (1)-(3). This process represents locating a crown center, using the coordinate system, for a tooth of the group of teeth.

FIG. 3B is an example step 340 in the aspect of FIG. 3A of the present simulation of attachments and aligners in accordance with this disclosure. In the example step 340, one axial plane XY is assumed and representation of incremental directional force system changes 342 is provided for this axial plane. For the axial plane assumed, a correspondent force system may be determined as tipped 10 and 40 degrees 342A with respect to the X axis for a maxillary first molar 344. From this, different force and moment combinations 346 at the C_{RES} 348 are determined. The different force and moment combinations are drawn to an equivalence of seventeen single forces spaced 2 mm or 1 mm from each other, where the central force is applied at the C_{RES} 348. A higher resolution force increment (1 mm) may be applied in the region with M:F ration of [−4:4]. To vary directions, the force is constantly maintained, but its direction for each position may be changed in the spatial plane of interest in 10 degree increments, resulting in 10 different simulations for each M:F value at the C_{RES}.

Methods disclosed here are valid for every technique that measures the force system delivered to the tooth by an orthodontic appliance. In one example, simulation models may be directed to discretization of the moments and forces. As such, available simulation models include Finite Element Analysis (FEA), Finite Difference analysis, and Finite Volume analysis. In these methods differential equations, such as partial differential equations are converted to sets of polynomials with convergence when parameters in the partial differential equations are adjusted. The parameters are as discussed throughout this disclosure, including the moments and forces available at the C_{RES} and the C_{ROT}. This is readily apparent to a person of ordinary skill in the art upon reading the present disclosure. Through FEA, comparative maps of effects of relevant M:F combinations on each tooth can be generated. Then, statistical evaluation of the comparative maps is conducted to determine whether the tooth morphological features are able to predict how the tooth will move with a specific force system. For example, such statistical evaluation may be to find the best fit or dominant features in the tooth morphological features. The best fit or dominant features provides significance of the comparative maps—indicating to a professional that the biomechanical behavior of any tooth may be derived using the presently disclosed force system and tooth dimensions. Moreover, this may be done without further FEA requirements.

Subsequent to the above process, sets of predictors—as made available in the CBCT analysis—can be applied to a generic tooth instead of requiring a subject tooth, when the C_{RES} coordinates (e.g., location) are known for the subject tooth. As such, a relationship is established between the distance D_{C.Res-C.Rot} and the tooth shape and its dimensions. The Burstone formula, as previously described, provides this relationship for at least an incisor on the tooth long axis in 2D. The present statistical methods, therefore, establish a 3D mathematical relationship between the distance D_{C.Res-C.Rot} and tooth morphology, which may be translated to a 3D graphical model as illustrated in FIGS. 2-4. The C_{RES} coordinates are also analyzed for provided datasets from different subjects to introduce a set of predictors that may be determined from discriminant analysis to provide dominant features. The dominant features may then be used calculate the 3D C_{RES} coordinates based on the tooth dimensions, which translates the 2D data to 3D. The predictors obtained by such statistical analysis of the C_{RES} locations may be for prescribed numbers of maxillary and/or mandibular teeth, such as, e.g., 14 maxillary and 14 mandibular teeth, and as more data is collected, accuracy of the system can be improved due to increases in the sample size.

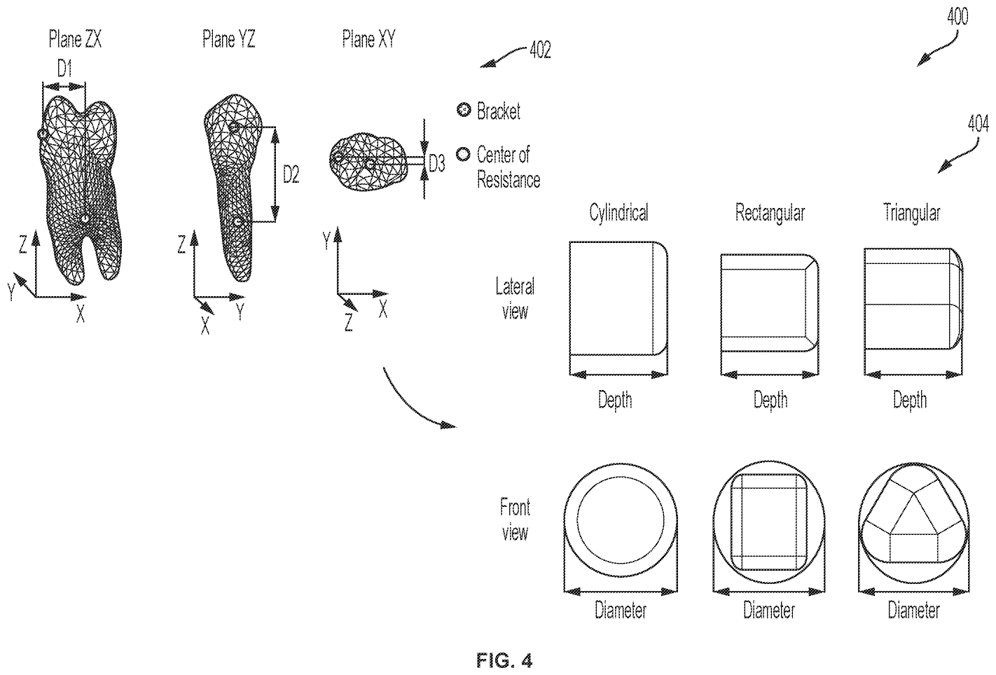

FIG. 4 is yet another aspect of an example process flow 400 for performing the present simulation of attachments and aligners 404 in accordance with this disclosure. Particularly, FIG. 4 provides simulated attachments 404 available for simulated aligners that may be a basis to generate physical format attachments and/or aligners for subject requiring orthodontic correction. A distance between a predicted or an expected C_{ROT} 402 and the actual C_{ROT} can be evaluated through equation (5):

$$D = \sqrt{{( \frac{{k}_{Fx}}{\frac{Mz}{Fx}} - \frac{{k}_{Fx}}{{MF}_{1}} )}^{2} + {( \frac{{k}_{Fy}}{\frac{Mz}{Fy}} - \frac{{k}_{Fy}}{{MF}_{2}} )}^{2}} \qquad Eq . ( 5 )$$

Here, MF₁ and MF₂ represent the M:F ratios necessary to obtain the optimal C_{ROT} coordinate on the plane and can be evaluated knowing k and the distance between the C_{RES} and the expected C_{ROT}, as discussed with respect to equation (4). The k value being specific for each tooth, as discussed with reference to TABLE 1, and being dependent on the tooth morphology, then requires specific one of the attachments 404. For the required M:F ratios to achieved the expected C_{ROT} 402, different attachments 404 are available. In a further example, results of average of different scenarios (M:F ratios with variations based on k and expected C_{ROT} 402) for each attachment shape may be calculated and used to rank the different configurations. TABLE 3 provides example M:F ratios for a rotation along the tooth long axis.

TABLE 3

| | | Fx | Fy | Fz | Mx | My | Mz | Mz/Fx | Mz/Fy |
|---|---|---|---|---|---|---|---|---|---|
| No | No | −0.04 | 0.00 | 0.14 | 0.03 | −0.01 | −2.09 | 55.00 | −1306.25 |
| Occlusal | attachment | | | | | | | | |
| load | Cylinder | 0.00 | 0.00 | 0.08 | 0.15 | −0.31 | −6.33 | −7912.50 | −1758.33 |
| | Rectangular | 0.00 | 0.00 | 0.15 | −0.35 | −0.69 | −6.19 | 3097.00 | 5630.91 |
| | prism | | | | | | | | |
| | Triangular | −0.05 | −0.01 | 0.21 | −0.91 | −1.11 | −5.85 | 119.39 | 603.09 |
| | prism | | | | | | | | |
| | Simulated | −1.2 | 0.5 | 1.7 | −5.8 | −8.8 | −13.2 | 11 | −26.4 |
| | Transducer | | | | | | | | |
| Occlusal | No | −0.06 | −0.09 | 0.87 | 0.84 | 0.10 | −4.70 | 82.46 | 52.81 |
| Load | attachment | | | | | | | | |
| | Cylinder | 0.08 | −0.08 | 1.12 | −0.81 | −4.61 | −7.72 | −91.90 | 98.97 |
| | Rectangular | 0.07 | −0.15 | 0.80 | −0.78 | −1.90 | −8.89 | −134.70 | 59.27 |
| | prism | | | | | | | | |
| | Triangular | 0.07 | −0.16 | 0.86 | −0.29 | −1.79 | −8.39 | −129.02 | 52.41 |
| | prism | | | | | | | | |

Based in part on the M:F ratios, such as from TABLE 3, a determination of the resulting distances of movement expected using the attachments are made. An example set of resulting distances are provided in TABLE 4. One of ordinary skill would understand from the present disclosure that a cylindrical-shaped attachment provides best result in both scenarios in these examples.

TABLE 4

| | No Occlusal | Occlusal | |
|---|---|---|---|
| | constraint | constraint | Average |
| No | 0.09 | 0.23 | 0.16 |
| attachment | | | |
| Cylinder | 0.005 | 0.14 | 0.07 |
| Rectangular | 0.002 | 0.18 | 0.09 |
| prism | | | |
| Triangular | 0.044 | 0.21 | 0.12 |
| prism | | | |
| Simulated | 0.55 | N/A | 0.55 |
| Transducer | | | |

From the prior determination of an attachment at the crown location for a digital aligner, a physical format attachment for an aligner may be provided for the group of teeth. Furthermore, an aggregate measure may be utilized with the above measurements to determine how many teeth require the attachments to achieve the desired rotation and movement. Further, such measurements may be parametrically changed to evaluate the effect of each variable on the efficacy of the desired orthodontic movement prior to finalizing the position of the brackets or attachments for the physical format aligners. The selection of a cylinder with optimum dimensions results in better movement than some of the other example attachments 404. The results in TABLE 4 support that the shape of the attachment is an important component of the present disclosure. For example, the above TABLE 4 provides that there are least constraints to a cylinder shape. The cylinder shape does not load the tooth as much with undesirable force systems, and limits the loads to the tooth with the desired force system. This may be because of a smaller distance between the intended C_{ROT} and the actual for the tooth C_{ROT} for the tooth. As such, the magnitude of the force system in undesirable components that could lead the tooth to an undesired position is also small because of the distance of movement being small.

Furthermore, in some embodiments, the distance between the resulting C_{ROT} and the expected C_{ROT} on each plane can be determined, e.g., after measuring the force system delivered to a selected tooth, through the following set of equations:

$$\begin{matrix}PLANE XY & \\ Dxy = \sqrt{{( \frac{{k}_{Fx}}{\frac{Mz}{Fx}} - \frac{{k}_{Fx}}{{MF}_{xy 1}} )}^{2} + {( \frac{{k}_{Fy}}{\frac{Mz}{Fy}} - \frac{{k}_{Fy}}{{MF}_{xy 2}} )}^{2}} & Eq . ( 6 )\end{matrix}$$

$$\begin{matrix}PLANE YZ & \\ Dyz = \sqrt{{( \frac{{k}_{Fy}}{\frac{M x}{Fy}} - \frac{{k}_{Fy}}{{MF}_{yz 1}} )}^{2} + {( \frac{{k}_{Fz}}{\frac{M x}{Fz}} - \frac{{k}_{Fz}}{{MF}_{yz 2}} )}^{2}} & Eq . ( 7 )\end{matrix}$$

$$\begin{matrix}PLANE ZX & \\ Dzx = \sqrt{{( \frac{{k}_{Fz}}{\frac{M y}{Fz}} - \frac{{k}_{Fz}}{{MF}_{zx 1}} )}^{2} + {( \frac{{k}_{Fx}}{\frac{M y}{Fx}} - \frac{{k}_{Fx}}{{MF}_{zx 2}} )}^{2}} & Eq . ( 8 )\end{matrix}$$

Where:
MF_{xy1}=Optimal expected Mz/Fx
MF_{xy2}=Optimal expected Mz/Fy
MF_{yz1}=Optimal expected Mx/Fy
MF_{yz2}=Optimal expected Mx/Fz
MF_{zx1}=Optimal expected My/Fz
MF_{zx2}=Optimal expected My/Fx

Using the distances resulting by the Eqs. 6-8 may indicate how much the force system elicited by the appliance differs from the optimal force system making it easier to compare results obtained by different appliances or attachments.

FIG. 5 provides example aspects of alignment 500 available via the example simulation of attachments and aligners 506 in accordance with this disclosure. The present method and system can be used with average dimensioned teeth from a general set of teeth or with dimensions from a subject's teeth obtained via scans as discussed with respect to FIGS. 1 and 2. Either data—from the average dimensioned teeth or dimensions from a subject's teeth—can be used to simulate the initial phase of an orthodontic treatment with aligners on a full maxillary arch. When dimensions from a subject's teeth are used, measurement values for average dimensioned teeth are substituted with measurement values from the subject's teeth using a merging of results from a CBCT and of an optical scan of the crowns of the subject's teeth. This ensures that a physical format aligner and related attachments, generated from finalized simulations, have a customized model for the subject. To achieve the finalized simulations, at least one variation to the attachment or the digital aligner is provided. The at least one variation is based on digital penetration between the aligner and the subject's teeth. Such a process may be taken as a substitute for physical changes that may require subsequent visits by the subject to adjust a physical format aligner, for instance.

The above rationale achieves a similar result as changing the aligner shape for each subject during a phase of achieved movement in a physical alignment process, but has at least an advantage of having faster setup times, reducing the number of subsequent visits, and simulating different movements that may not be observed between scheduled visits. The present disclosure, thereby, predicts the correction and reduces the number of subsequent adjustments required by a subject. The use of the above-referenced mathematical processes achieves equilibrium to the same endpoint—correction of a subject's teeth alignment. FIG. 5 particularly provides basic shape and dimensions 504 (e.g., depth 508 and diameter 510) for the attachment. Example movements 502 provide the above-referenced movements and rotation available and desired in the process for reposition, correcting, and aligning a tooth and one or more associated teeth. Mesio-Distal and Linguo-Buccal torques are obtained by movements around the Mesio-Distal and the Linguo-Buccal axes, respectively. The rotation is obtained when the tooth rotates around its center as illustrated in example movements 502.

FIGS. 6A and 6B are graphical representations of the torque quantity and torque quality obtained for different depths of attachments and aligners in accordance with this disclosure. FIGS. 6C and 6D are graphical representations of the torque quantity and torque quality obtained for different diameters of attachments and aligners in accordance with this disclosure. FIGS. 6E and 6F are graphical representations of the rotation quantity and rotation quality obtained for different depths of attachments and aligners in accordance with this disclosure. FIGS. 6G and 6H are graphical representations of the rotation quantity and rotation quality obtained for different diameters of attachments and aligners in accordance with this disclosure.

FIGS. 6A-6H illustrate example graphical variations 600 and 640 available via the example simulation of attachments and aligners in accordance with this disclosure. FIGS. 6A-6D illustrate graphical variations 600 obtained for different attachments with different shapes and sizes. The graphical variations 600 provide different torque quantities obtained for different depths and diameters of the attachments—as in the example attachment 506 in FIG. 5. The variations 600 also provide the torque quantity separated by a quantity and quality of the parameters applied. The torque quality, as used herein, refers to the distance between the actual C_{ROT} and the expected (intended and/or desired) C_{ROT}, using the optimal C_{ROT}. The torque quantity, as used herein, refers to an amount of expected (intended and/or desired) Moment achieved using the force system determined for the C_{RES} at issue. The variations graphs also illustrate trends while varying the attachment depth or diameter. The no attachment implementation does not reflect in the quality graphs because the resulting distance given by D_{C.Res-C.Rot} is larger than the available scale provided to illustrate the distance obtained using the other shapes.

FIGS. 6E-6H provides graphical variations 640 obtained for different attachments of different shapes and sizes. As in the case of FIGS. 6A-6H, the graphical variations 640 are provided in terms of rotation quantity and rotation quality. Rotation quality, as used herein, is the distance from C_{ROT} and an optimal expected C_{ROT}, while rotation quantity, as used herein, refers to an amount of desired Moment achieved. The graphical variations 640 allow evaluation of the effects of intended C_{ROT} in the singular plane—there is no intended C_{ROT} in other axial planes as previously noted. Moreover, the graphical variations 640 illustrate trends while changing the attachment depth or diameter—as in the example attachment 506 in FIG. 5. The graphical variations 640 illustrate that the effect of diameter is most significant on force system rotation quantity, and also affects the rotation quality in the rectangle at the cost of increasing effects.

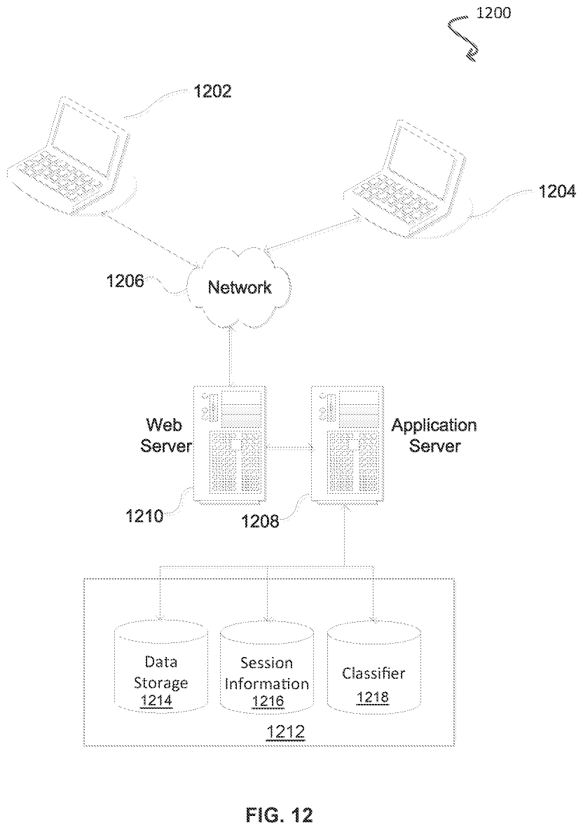

For this example, the graphical variations 600 and 640 of FIGS. 6A-6H provide that a triangle attachment results in a best attachment for the mesio-distal torque. This is because the simulated aligner (and by virtue, the physical format aligner) requires a level of grip for the attachment to offer a rotational degree of freedom for the best movement. FIGS. 61, 6J, and 6K are graphical representations of the deviation of the rotation axis, moment Z-axis, and the C_{ROT} deviation for different depths of attachments and aligners in accordance with this disclosure. FIGS. 6L, 6M, and 6N are graphical representations of the deviation of the rotation axis, moment Z-axis, and the C_{ROT} deviation for different diameter of attachments and aligners in accordance with this disclosure. FIGS. 60, 6P, and 6Q are graphical representations of the deviation of the rotation axis, moment Z-axis, and the C_{ROT} deviation for different mesiodistal distance from the crown center for an attachment in accordance with this disclosure.

FIGS. 7A-7Q illustrate example graphical comparisons 700, 740, and 760 between predicted and tested values of a k parameter and force direction for simulation of attachments and aligners and for physical attachments and aligners in accordance with this disclosure. Particularly, FIGS. 7A-7N provide graphical comparisons 700 and 740 of parameter k against values of force direction measured for different types of teeth on different planes, in an example from real patients. FIGS. 7A and 7B are graphical representations of parameter k against values of force direction measured for maxillary and mandibular central incisors, respectively, on different planes, in an example from real patients. FIGS. 7C and 7D are graphical representations of parameter k against values of force direction measured for maxillary and mandibular lateral incisors, respectively, on different planes, in an example from real patients. FIGS. 7E and 7F are graphical representations of parameter k against values of force direction measured for maxillary and mandibular canines, respectively, on different planes, in an example from real patients. FIGS. 7G and 7H are graphical representations of parameter k against values of force direction measured for maxillary and mandibular first premolars, respectively, on different planes, in an example from real patients. FIGS. 71 and 7J are graphical representations of parameter k against values of force direction measured for maxillary and mandibular second premolars, respectively, on different planes, in an example from real patients. FIGS. 7K and 7L are graphical representations of parameter k against values of force direction measured for maxillary and mandibular first molars, respectively, on different planes, in an example from real patients. FIGS. 7M and 7N are graphical representations of parameter k against values of force direction measured for maxillary and mandibular second molars, respectively, on different planes, in an example from real patients. This example is provided to illustrate that M:F ratios share a predictable relationship with C_{ROT}. For example, in each of type of tooth, the k parameter rises with the force direction in the YZ plane, while the k parameter decreases or presents ineffective values as the force direction increases. Particularly, in FIGS. 7A-7N, shapes for the k parameter (e.g., area under the curve) for increased greatly when the force direction became parallel to the tooth long axis (Z-axis) and shapes for the k parameter were larger on the YZ and ZX planes than on the XY plane which contained the Mesio-Distal and Bucco-Lingual dimensions of the teeth. In addition, these shapes are also available to predict the type of tooth as the shape of the curves are substantially the same for similar tooth types and different shapes are indicative of different teeth.

Further, as indicated in FIG. 7R, the M:F ratios and the D (C_{RES}-C_{ROT}) can have an hyperbolic relationship in every tooth, characterized by the parameter k, which changes for each tooth and each force system. FIG. 7R shows a graphical representation of a relationship between the moments and forces (M:F) and the distance between C_{ROT} and C_{RES} (“D_{C.Res-C.Rot}”) for an example tooth and an example angle in accordance with this disclosure. As such, for the planes YZ and ZX, parameter k is always higher when the force direction is congruent with Z-axis which represents the tooth long axis. In addition, the parameter k has an average increase of 329% for maxillary teeth and 313% for mandibular teeth, when the force changed from parallel to Y axis to parallel to z axis on the plane YZ. Furthermore, the average increase moving from axis X to Z on the plane ZX was 203% for maxillary teeth and 248% mandibular teeth. On plane XY, for the maxillary teeth, the average increase is 87% and for the mandibular teeth, the average increase is 79%. The same trend appears for axial planes on both maxillary and mandibular arches.

FIGS. 70, 7P, and 7Q are graphical representations of the predicted and calculated values of the k parameter for simulation of attachments and aligners and for physical attachments and aligners in the XY plane, YZ plane, and ZX plane, respectively, in accordance with this disclosure. FIGS. 70, 7P, and 7Q further illustrates a comparison 760 comparison of k values predicted based on tooth morphology, with k being calculated for a sample tooth. The k parameters, on the different planes, are determined to depend on the moment direction in addition to the force direction of FIGS. 7A-7N. When force is applied parallel to the Mesio-Distal axis (Y-axis) the k parameters have an average value of 3.3 with the moment applied along the Z-axis (Plane XY), but has an average value of 15.1 when the moment is applied along the z-axis (Plane YZ). This comparison confirms that k parameters are predictable with the set of predictors, such as the Bucco-Lingual dimension at the CEJ; the Mesio-Distal dimension at CEJ; the root length on the plane YZ; the root length on the plane ZX; and the average between the root lengths on the planes YZ and ZX. The predictors used to calculate the k parameters are unstandardized coefficients, but the present disclosure makes it possible to utilize the predictors along with generalized or specific tooth dimensions for each tooth and for each planes and hence.

FIG. 8 illustrates examples 800 of penetration values and digital penetration obtained from movement, in the example simulation, between attachments and aligners and a tooth, along with adjacent teeth, in accordance with this disclosure. To conduct the present simulation, a general set of teeth may be generated with artificial periodontal ligament (PDL) based on input dimensions from a dataset of prior patients. The PDL for each tooth may be created/generated, e.g., using CAD modelling as will be readily understood by a person of ordinary skill reading this disclosure. For example, each maxillary and mandibular tooth may be modeled from a population subset of data of real patients. The model may then be sliced to obtain Tooth-PDL-Bone multibody models from each arch for the Finite Element Analysis. While this may require in-vivo validation, computer-based simulations using material properties extensively support the underlying simulation to provide replication that is very similar to physical sets of teeth. A simulation model, such as FEA can simulate such a complex structure. Bone and teeth portions may be modeled as simplified homogenous bodies, without discerning between cortical and cancellous bone and enamel or pulp and dentin. This allows for saving computational time. A linear elastic model may be used for each model structure to test the movements under the assumption of low PDL strains, e.g., less than (<) about 7.0% to about 8.0%, such as about 7.5% in one embodiment. If the PDL strain exceeds 7.5%, non-linear models can be used for testing of the movements.

The equilibrium equation solving capability in the FEA enables resolution of the above modeling implement our realistic testing method. The FEA simulation model also allows for continuous optimization of the design of these appliances simulating a multitude of oral conditions. FIG. 8 particularly illustrates the application of load in the FEA simulation model to generate the orthodontic movement for digital penetration. This is a manner of simulating interaction between teeth and aligner without a need for a subject to wear a physical aligner. As such, a wearing phase of the aligner to the teeth, until equilibrium is reached, is simulated in present FEA simulation model. For example, for the digital model, the aligner is digitally positioned over the teeth at time=0 and is slowly moved towards the dentition and deformed to fit over them. The load which allows the teeth movement is the aligner elastic deformation, which during the relaxation phase time=1 forces the teeth to the new equilibrium position (time=2). For the FEA simulation model, the aligner and the target tooth have an initial penetration representing the load for the FEA simulation model, as discussed with regards to the prior figures, and a solution for the FEA simulation model is reached when equilibrium is reached, thereby providing the digital penetration. In FIG. 8, initial penetration between aligner and tooth for the rotation of a maxillary first premolar is illustrated.

FIGS. 9A, 9B, 9C, 9D, and 9E provide example flow diagrams 900, 930, 960, 980, and 990 of methods/processes to perform the example simulation of attachments and aligners in accordance with this disclosure. FIGS. 9A, 9B, 9C, 9D and 9E illustrate example methods 900, 930, 960, 980, and 990 applicable in a system including at least one processor and memory including instructions is disclosed. The instructions, when executed in part by the at least one processor, cause the at least one processor to support or perform certain function as disclosed in these figures. In addition, the methods 900, 930, 960, 980, and 990 may be configured into or used by the system as disclosed with regard to FIGS. 11 and 12, or into any system configured in the manner as disclosed herein is also discussed. Furthermore, a non-transitory medium including instructions for executing on at least one processor is available to enable any system to support or perform the functions in these figures.

For example, method 900 includes a sub-process 902 for determining axial plane values for at least a group of teeth. A coordinate system is determined, via sub-process 904, from the axial plane values. Sub-process 906 allows locating of a crown location, such as a crown center or other suitable crown location, using the coordinate system. The crown location, such as the crown center, is for a tooth of the group of teeth. The tooth may be a target tooth that requires the most movement in an aligning process for a patient. In sub-process 908, an attachment at the crown center for a digital aligner is determined. The attachment at the crown center for the digital aligner may be determined for provision a physical format for the group of teeth. In sub-process 910, a digital penetration is determined between the digital aligner and the tooth. A simulation model, such as an FEA, may be used via sub-process 912, to determine orthodontic data for a center of rotation for the tooth based at least in part on the digital penetration. Sub-process 914 provides verification for whether at least one variation to the attachment or the digital aligner exists based in part on the orthodontic data. When the verification cannot confirm an existence of a variation, then sub-process 902 may be initiated with other axial plane values. When the verification confirms a variation to the attachment or the digital aligner, this variation is provided based in part on the orthodontic data, via sub-process 916. The variation reflects equilibrium achieved for the digital penetration after the initial digital penetration illustrated in FIG. 8.

Furthermore, the sub-process 912 may calculate the center of rotation for the tooth based at least in part on the digital penetration. This reflects a change in the center of rotation as the simulated wearing phase has occurred. The method 900 also supports generating a digital view of the digital aligner in an initial configuration as provided in FIG. 8. Furthermore, as discussed throughout this disclosure, a new digital view may be generated for the digital aligner in a new configuration after application of the at least one variation. In a further implementation, the aligner is created through Boolean operations to be a 0.7 mm shell completely congruent with the teeth shape based in part on the FEA simulation model using the initial values. In an embodiment, it can be substituted with a comparatively more realistic non-uniform aligner. In certain embodiments, the aligner is created by CAD modelling or 3D imaging. Afterwards, the target teeth can be moved from their original position to create digital penetration between aligner and teeth. This reversed rationale achieves the same result as changing the aligner shape for each scenario but has the advantage of quicker setups to simulate different movements, because mathematically equilibrium is resolved to the same endpoint. Then, the aligner or attachment modification is tested to verify how well the tooth is taken to the normal position.

The orthodontic data is determined for each simulation are the M:F values at the center of resistance of the tooth and distances between the intended and actual (or obtained) centers of rotation. The simulated attachments for the simulated aligners are tested for digital penetration. In an example, a pre-built library of tooth movements and force systems, along with one or more statistical models may be used to individualize the relationships between centers of rotation and force systems for any tooth of any subject from the subject's basic tooth dimensions. The at least one variation may be one or more of: a shape or a material for a physical aligner corresponding to the digital aligner. In another example, the method 900 may include further sub-processes for applying one or more of: a Cone Beam Computed Tomography (“CBCT”) and an optical scan for a subject; and for determining the axial plane values for at least the group of teeth from the one or more of the CBCT and the optical scan. In yet another implementation, the method 900 includes additional features for generating averaged dimensioned teeth for a subject. The method 900 may then determine the axial plane values from the averaged dimensioned teeth.

In addition to the above, method 900 may further include features to determine that one of: load magnitudes and displacements, for the tooth, is affected by a shape or a material proposed in the digital aligner. When such a determination is made then a further determining may be performed for one of: stiffness, diameter, depth, and position and orientation on the crown of the digital aligner that may be varied as part of the at least one variation to the attachment or the digital aligner. A selecting sub-process is performed for a configuration including a selected one of: load magnitudes and displacements, and a selected one of: stiffness, diameter, and depth, for the attachment or the digital aligner.

FIG. 9B presents method 930 that may be performed independently of method 900 or subsequently, and even, concurrently with method 900. In method 930, sub-process 932 determines anatomical structure using CBCT from a subject or from a CAD model of averaged dimensioned teeth for the subject. A determination is processed via sub-process 934 for coordinate systems according to one or more occlusal plane for at least a tooth of a group of teeth. A locating feature of sub-process 936 locates a crown center using one or more of the coordinate systems. The crown center is for the tooth that needs movement. Sub-process 938 determines one or more attachments at the crown center for a digital aligner for the tooth. A generating feature in sub-process 940 generates a digital penetration including at least a movement between the digital aligner and the tooth. Furthermore, in sub-process 942 a determination occurs for orthodontic data for a center of resistance for the tooth based at least in part on the digital penetration. The orthodontic data refers to at least a k parameter, C_{RES}, C_{ROT}, and distance D) required to generate the simulation model. A verification of an availability of the simulation model for the orthodontic data is performed via sub-process 944.

When no simulation model is available, further data is sought via sub-process 932. When the orthodontic data is sufficient and suitable, a generation of the simulation model occurs, via sub-process 946, using the orthodontic data. A determination for moments and forces at the center of resistance based in part on the simulation model is made via sub-process 948. A second verification is performed—this time for the simulation model to reflect at least one variation to the attachment or the digital aligner. This is performed via sub-process 950. A failure of the verification would initiate a further simulation model, perhaps with variation to the orthodontic data. When the verification passes, a change to the attachment or the digital aligner is provided using the at least one variation, via sub-process 952.

FIG. 9C presents method 960 that may be performed independently of methods 900, 930 or subsequently, and even, concurrently with method 900, 930. In method 960, sub-process 962 performs a determination that a simulation model provides different moments and different forces at a center of resistance based in part on orthodontic data from digital penetration including at least a movement between the digital aligner and a tooth of a group of teeth. Sub-process 964 performs a determination for configurations of the moments and the forces based at least in part on a comparison of load magnitude and displacements generated from one or more different coordinate systems according to one or more occlusal plane. In sub-process 966 a further determination occurs, for an individual configuration of the configurations, one or more of: stiffness, diameters, and depths, each contributing to at least one variation for the simulation model. Application the at least one variation to update the simulation model with and without occlusal loads occurs via sub-process 968. Sub-process 970 generates updates to at least one attachment of a digital aligner for the tooth based in part on the update to the simulation model. In sub-process 972, a determination occurs for updated moments and updated forces at the center of resistance based in part on the update to the simulation model. A verification sub-process 974 performs a check for any further updated moments and further updated forces for the configurations. When it is determined that there are updates, sub-process 966 starts. When there are no updates, a provisioning sub-process 976 provides, from the configurations, a selected configuration representing a predictability value exceeding a threshold.

FIG. 9D presents method 980 that may combine or be independent of features from methods 900, 930, and 960. Method 980 is sectioned into sub-methods 982, 984, and 986. Sub-method 984 is a loop to incorporate all possible force directions for the simulation model. In method 980, integration of CBCT and optical scan is performed via sub-process 982A. The integration creates CAD models of tooth and bone. Sub-process 982B creates the PDL using Boolean operations.

In an example, TABLE 5 is generated from 3D models for each tooth-PDL-bone complex by integrating CBCT scans obtained for the subjects using Planmeca® ProMax® 3D Max unit and surface structured light scan. An optical scanner was used to reconstruct the teeth crowns through the digitalization of plaster casts. The 3D individual dental tissues obtained by the optical scanner and the CBCT were fused to obtain multi-body orthodontic models with minimum user interaction. The obtained geometries were auto patched to create trimmed Non-uniform rational basis spline (NURBS) surfaces, which were converted into vendor neutral file format allowing the exchange of CAD models Initial Graphics Exchange Specification (IGES) using Geomagic Studio®. The points of measurement for the tooth dimensions of TABLE 5 are illustrated in FIG. 2B.

TABLE 5

| | | | | Root | | |
|---|---|---|---|---|---|---|
| | | | | length | Root | |
| | | | | plane | length | Root |
| | | MD | LB | YZ | plane ZX | AVG |
| | | (mm) | (mm) | (mm) | (mm) | (mm) |
| Subject | Maxillary central | 6.1 | 5.8 | 15.6 | 16.2 | 15.9 |
| 1 | incisor | | | | | |
| | Maxillary lateral | 4.2 | 5.8 | 15.1 | 15.5 | 15.3 |
| | incisor | | | | | |
| | Maxillary canine | 6.2 | 8.8 | 15.8 | 16.0 | 15.9 |
| | Maxillary first | 4.9 | 8.7 | 13.6 | 14.2 | 13.9 |
| | premolar | | | | | |
| | Maxillary second | 5.4 | 8.4 | 14.4 | 14.3 | 14.4 |
| | premolar | | | | | |
| | Maxillary first | 7.2 | 9.0 | 13.1 | 12.8 | 13.0 |
| | molar | | | | | |
| | Maxillary second | 7.8 | 10.1 | 12.5 | 12.0 | 12.3 |
| | molar | | | | | |
| | Mandibular | 4.3 | 6.2 | 13.1 | 14.0 | 13.6 |
| | central incisor | | | | | |
| | Mandibular | 3.9 | 6.3 | 12.2 | 12.9 | 12.6 |
| | lateral incisor | | | | | |
| | Mandibular | 3.9 | 5.9 | 12.5 | 14.0 | 13.3 |
| | canine | | | | | |
| | Mandibular first | 4.8 | 6.9 | 10.8 | 11.7 | 11.3 |
| | premolar | | | | | |
| | Mandibular | 4.9 | 7.0 | 13.8 | 14.2 | 14.0 |
| | second premolar | | | | | |
| | Mandibular first | 9.2 | 9.5 | 11.8 | 12.0 | 11.9 |
| | molar | | | | | |
| | Mandibular | 9.2 | 8.2 | 11.6 | 12.1 | 11.9 |
| | second molar | | | | | |
| Subject | Maxillary central | 6.9 | 7.4 | 13.7 | 15.8 | 14.7 |
| 2 | incisor | | | | | |
| | Maxillary lateral | 4.8 | 6.4 | 12.0 | 13.6 | 12.8 |
| | incisor | | | | | |
| | Maxillary canine | 6.2 | 7.4 | 16.0 | 16.4 | 16.2 |
| | Maxillary first | 5.5 | 8.4 | 12.3 | 13.3 | 12.8 |
| | premolar | | | | | |
| | Maxillary second | 6.1 | 8.9 | 13.2 | 13.8 | 13.5 |
| | premolar | | | | | |
| | Maxillary first | 8.8 | 8.8 | 11.4 | 12.2 | 11.8 |
| | molar | | | | | |
| | Maxillary second | 9.2 | 10.8 | 13.2 | 12.8 | 13.0 |
| | molar | | | | | |
| | Mandibular | 3.8 | 5.6 | 11.4 | 12.1 | 11.8 |
| | central incisor | | | | | |
| | Mandibular | 3.5 | 5.5 | 10.6 | 11.5 | 11.1 |
| | lateral incisor | | | | | |
| | Mandibular | 3.5 | 5.3 | 10.9 | 12.1 | 11.5 |
| | canine | | | | | |
| | Mandibular first | 4.4 | 6.2 | 9.4 | 10.1 | 9.8 |
| | premolar | | | | | |
| | Mandibular | 4.3 | 6.3 | 12.0 | 12.3 | 12.2 |
| | second premolar | | | | | |
| | Mandibular first | 8.1 | 8.3 | 10.2 | 10.4 | 10.3 |
| | molar | | | | | |
| | Mandibular | 8.1 | 7.3 | 10.1 | 10.5 | 10.3 |
| | second molar | | | | | |

A linear elastic model is applied for each structure to test the movements under the assumption of low PDL strains as in TABLE 6.

TABLE 6

| Structure | Young Modulus (MPa) | Poisson's ratio |
|---|---|---|
| Tooth | 20.000 | 0.3 |
| Bone | 13.000 | 0.3 |
| PDL | 0.059 | 0.3 |

The geometries from TABLE 5 and any required material specification from TABLE 5 or other information are fed into a simulation model, such as an FEA, where all the bodies were meshed with solid elements. The coordinate system was defined for each tooth according to the occlusal plane. The X-axis was congruent with the Linguo-Buccal tooth dimension, while the Y-axis with the Mesio-Distal and the Z-axis was perpendicular to the occlusal plane. An example of coordinate system is shown in FIG. 2B, by example 212.

Sub-process 982C illustrates a flow of the feeding of the CAD models into an FEA solver, such as Ansys16®. The FEA solver seeks to evaluate the parameters entered to provide a simulation model that is then subject to configuration changes to determine digital penetration. Sub-process 982D provides definition of the coordinate system according to the occlusal plane in the manner performed in methods 900, 930, and 960. Further, definition, as used in this example is a determination process for the coordinate system using the occlusal plane. Sub-process 982E evaluates a C_{RES} coordinate from the provided definitions. The loop sub-method 984 then occurs. In sub-process 984A, force application occurs according to TABLE 7, which lists moment and forces applicable to each tooth to locate the approximate C_{RES}, while keeping the PDL principal strain value below 7.5%.

TABLE 7

| | | Central | Lateral | | First |
|---|---|---|---|---|---|
| | | Incisor | incisor | Canine | Premolar |
| Moment | Maxillary | −1.8 | −1.08 | −1.68 | −1.56 |
| (Nmm) | Mandibular | −1.08 | −1.08 | −1.44 | −1.44 |
| Force (N) | Maxillary | 0.15 | 0.09 | 0.14 | 0.13 |
| | Mandibular | 0.09 | 0.09 | 0.12 | 0.12 |

| | | Second | First | Second |
|---|---|---|---|---|
| | | Premolar | Molar | Molar |
| Moment | Maxillary | −1.56 | −2.88 | −3 |
| (Nmm) | Mandibular | −1.56 | −2.76 | −2.28 |
| Force (N) | Maxillary | 0.13 | 0.24 | 0.25 |
| | Mandibular | 0.13 | 0.23 | 0.19 |

The C_{RES} is found for three configurations of each tooth-one for each coordinate plane, by applying a moment with the specific values shown in TABLE 7. A tooth-specific constant force was applied at the C_{RES}, while the M:F was varied from about-12 mm to about 12 mm, for each tooth in TABLE 7. The moment amount as well as the forces amounts were proportionally different for each tooth, because each tooth requires a different load to keep the PDL strain <7.5% and allowing for a linear PDL model within this range. The bone's nodes further can be assigned zero displacement to simulate a rigid body due to the transient nature of tooth displacement solely attributed to bone deformation. Simulations further can be performed on the three spatial planes (plane XY, plane YZ, plane ZX). In one example, for each plane, 17 equivalent force systems can be applied at the C_{RES} of each tooth, as shown in FIG. 3B.

Furthermore, the approximate 2D projections of the axes of resistance (from axis including C_{RES}) for each of the configurations are recorded. A mesh defining each region of the digital aligner is iteratively generated until reaching an edge size of 0.1 mm is reached. This may be a maximum accepted error on the C_{RES} location. Final positions of the axes of resistance are recorded. Then, as noted in the above sub-processes, including sub-process 984B, for each tooth the average of three different C_{RES} configurations, obtained at the last iteration, are evaluated and used as the approximate C_{RES} location for the subsequent analyses or changes to the simulation model. The C_{RES} coordinates were measured with the coordinate system located at the center of the CEJ (Cement-enamel-junction) as shown in FIG. 2B by example 216. This information is provided in TABLE 8.

TABLE 8

| | | | | | Loc z | Loc z | |
|---|---|---|---|---|---|---|---|
| | | | | | from | from | |
| | | | | | plane | plane | Average |
| | | | Loc x | Loc y | YZ | ZX | Loc z |
| | | Tooth | (mm) | (mm) | (mm) | (mm) | (mm) |
| Dataset | Maxillary | ur1 | 1.3 | −0.4 | −5.8 | −6.4 | −6.1 |
| 1 | | ur2 | 0.9 | −0.2 | −5.7 | −6.1 | −5.9 |
| | | ur3 | 1.4 | −0.4 | −6.2 | −6.4 | −6.3 |
| | | ur4 | 1.6 | −0.5 | −5.4 | −6.0 | −5.7 |
| | | ur5 | −0.5 | −0.3 | −5.4 | −5.3 | −5.4 |
| | | ur6 | −0.5 | 0.3 | −5.0 | −4.7 | −4.9 |
| | | ur7 | 1.1 | 1.3 | −4.7 | −4.2 | −4.5 |
| | Mandibular | lr1 | 0.4 | −0.2 | −5.2 | −6.1 | −5.7 |
| | | lr2 | 0.2 | 0.0 | −5.0 | −5.7 | −5.4 |
| | | lr3 | 0.2 | 0.1 | −5.8 | −7.3 | −6.6 |
| | | lr4 | 0.4 | 0.1 | −4.7 | −5.6 | −5.2 |
| | | lr5 | 0.9 | −0.6 | −4.9 | −5.3 | −5.1 |
| | | lr6 | 0.5 | −1.0 | −4.5 | −4.7 | −4.6 |
| | | lr7 | 1.7 | −0.2 | −4.2 | −4.7 | −4.5 |
| Dataset | Maxillary | ur1 | 0.0 | 0.0 | −5.2 | −7.3 | −6.3 |
| 2 | | ur2 | 0.6 | 0.0 | −4.6 | −6.2 | −5.4 |
| | | ur3 | −0.2 | −0.1 | −7.4 | −7.8 | −7.6 |
| | | ur4 | 1.6 | 0.6 | −5.3 | −6.3 | −5.8 |
| | | ur5 | 1.0 | −0.4 | −5.1 | −5.7 | −5.4 |
| | | ur6 | −0.3 | −0.3 | −4.0 | −4.8 | −4.4 |
| | | ur7 | 1.0 | −1.1 | −4.7 | −4.3 | −4.5 |
| | Mandibular | lr1 | 0.1 | 0.1 | −5.1 | −5.5 | −5.2 |
| | | lr2 | 0.4 | 0.2 | −4.0 | −4.9 | −4.5 |
| | | lr3 | −0.2 | −0.2 | −4.5 | −5.9 | −5.3 |
| | | lr4 | −0.3 | 0.0 | −3.6 | −4.3 | −4.0 |
| | | lr5 | 0.5 | −0.4 | −4.9 | −5.2 | −5.1 |
| | | lr6 | 0.4 | −1.2 | −4.1 | −4.3 | −4.2 |
| | | lr7 | 0.7 | −0.2 | −3.8 | −4.2 | −4.0 |

Sub-process 984C verifies if all force directions were evaluated. When such verification indicates that other force directions need evaluation, sub-process 984D is performed to rotate the force direction of 10 degrees towards the second coordinate axis and to perform sub-process 984B for updating the simulation model in the FEA. Sub-method 986, otherwise, occurs when all the force directions are evaluated. Sub-process 986A performs an application of the moment from TABLE 7 in a manner perpendicular to the plane. Loop of sub-method 986 may be repeated until the moments are exhausted as verified under sub-process 986B. When all the moments have no exhausted, sub-process 986D is performed prior to repeating the loop of sub-method 984. Sub-process 986C provides the evaluation of all of the C_{ROT} coordinates.

In an example, for each tooth, the different M:F were applied at the respective C_{RES}, via sub-processes 984A and 986A, to provide a generalized map of tooth movements referenced at the tooth that can be transferred to any appliance (e.g. brackets, aligners, etc.). This could be via equations (1)-(3) previously discussed. With the application of simple equivalent force system calculations, as in these equations, it is possible to transfer the force system to any bracket position.

The analysis process in each of methods 900, 930, 960, and 980 demonstrate that the C_{RES} coordinates can be calculated with a set of predictors shown in TABLE 9 with a significance of 0.05. The coefficients are noted as unstandardized and standardized (via short form) where appropriate.

TABLE 9

| | | | | | | 95.0% | |
|---|---|---|---|---|---|---|---|
| | | | | | | Confidence | |
| | | | Unstandardized | | | Interval for B | |
| | | | Coefficients | Std. | | Lower | Upper |
| Coordinate | Arch | Coefficient | B | Error | Sig. | Bound | Bound |
| X | Mandibular | (Constant) | .023 | 1.043 | .983 | −2.273 | 2.318 |
| | | MD (mm) | 186 | 141 | .214 | −.125 | .496 |
| | | LB (mm) | −.090 | .253 | 730 | −.647 | .468 |
| | Maxillary | (Constant) | 733 | 1.213 | 558 | −1.936 | 3.403 |
| | | MD (mm) | −.285 | 197 | .177 | −.720 | 150 |
| | | LB (mm) | .210 | .196 | 306 | −.221 | 642 |
| Y | Mandibular | (Constant) | 1.493 | .461 | .007 | .489 | 2.497 |
| | | LB (mm) | −.258 | .067 | 002 | −.404 | −.111 |
| | Maxillary | (Constant) | −.218 | .901 | 813 | −2.180 | 1.744 |
| | | LB (mm) | 014 | .108 | 896 | −.222 | .250 |
| Average Z | Mandibular | (Constant) | .112 | .764 | .886 | −1.569 | 1.793 |
| | | R_{YZ} (mm) | 793 | .224 | .005 | .301 | 1.286 |
| | | R_{ZX} (mm) | −1.164 | .209 | .000 | −1.625 | −.703 |
| | Maxillary | (Constant) | 1.529 | 1.104 | 194 | −.901 | 3.959 |
| | | R_{YZ} (mm) | 122 | .164 | .471 | −.238 | .482 |
| | | R_{ZX} (mm) | −.618 | .158 | 002 | −.965 | −.271 |

Different equations for each spatial coordinate discerns among maxillary and mandibular teeth as previously noted. The standard errors reported in TABLE 9 show that there is a lower relative error for the Z coordinate. This can be ascribed to the lower absolute value of the Z and Y coordinates which could have brought to lees precise measurements and maybe to a real lower correlation with the tooth morphology compared with the Z coordinate.

The analysis above confirms that parameter k is predictable and presently relied upon by inputting the root dimensions, the force direction, and the spatial plane, in a simulation mode, such as an FEA, with possible iterations to adjust at least one variation. The crown size was not statistically significant to predict parameter k. These results also show that a different set of predictors should be used for each plane and a further distinction should be based on the tooth morphology. One of ordinary skill, upon reading this disclosure can understand that a 95% confidence interval for the parameter k may be wide for some of the coefficients of this parameter, but that standard error depends on the number of samples and can be therefore reduced as more datasets are provided to the system.

As shown in TABLE 11, the “k” variability may be different among different teeth and among different planes for the same tooth. For example, for the maxillary lateral incisor of the first patient the percentage change varies from 42% on the plane XY to 366% on the plane YZ. While comparing the results on the same plane for different teeth, e.g., considering the plane XY for the first patient, the percentage difference varies from the 3.4% of the mandibular first molar to 151% of the maxillary second premolar. The analysis of the “k” values on the different planes may show that they depend not only on the force direction, but also on the moment direction. If the force is applied parallel to the mesio-distal tooth axis (y-axis) “k” assumes an average value of 3.3 if the moment is applied along the z-axis (Plane XY) and a value of 15.1 if the moment is applied along the z-axis (Plane YZ).

TABLE 10 shows exemplary datasets of “k” values for exemplary patients, e.g., patient 1 and patient 2:

TABLE 10

| | Central Incisor | | | Lateral Incisor | | | Canine | | | First Premolar | |
|---|---|---|---|---|---|---|---|---|---|---|---|
| Angle | XY | YZ | ZX | XY | YZ | ZX | XY | YZ | ZX | XY | YZ |

Patient 1

| | | | | | | | | | | | |
|---|---|---|---|---|---|---|---|---|---|---|---|
| Maxillary | | | | | | | | | | | |
| 0 | 2.7 | 21.3 | 77.5 | 3 | 12.1 | 33.9 | 6.1 | 21.7 | 63.2 | 8.8 | 14.3 |
| 10 | 2.5 | 30.2 | 73.4 | 2.9 | 14.3 | 32.9 | 6 | 32.9 | 57.6 | 8.7 | 18.7 |
| 20 | 2.3 | 43.4 | 67.2 | 2.8 | 16.3 | 31 | 5.7 | 47 | 53.8 | 8.5 | 25.1 |
| 30 | 2.2 | 57.8 | 59.9 | 2.6 | 17.4 | 28.7 | 5.4 | 57.7 | 48.6 | 8.1 | 31.5 |
| 40 | 2.1 | 70.6 | 53.5 | 2.4 | 23 | 25.2 | 5.1 | 67.2 | 43.8 | 7.5 | 37.5 |
| 50 | 2.2 | 80.4 | 47.7 | 2.2 | 30 | 21.4 | 5.3 | 79.7 | 38.7 | 6.8 | 43.2 |
| 60 | 2.4 | 88.8 | 42 | 2.1 | 37.8 | 18.3 | 4.6 | 89.6 | 32.9 | 6.1 | 48.2 |
| 70 | 2.7 | 92.2 | 38 | 2 | 44.8 | 15.8 | 3.7 | 104.6 | 27.4 | 5.5 | 52.3 |
| 80 | 3 | 95.3 | 35 | 2 | 51.3 | 11.8 | 3.4 | 115.3 | 25.2 | 5 | 56.1 |
| 90 | 3.5 | 93.4 | 34.2 | 2.1 | 56.4 | 11.7 | 3.2 | 119.4 | 23 | 4.9 | 58.7 |
| Mandibular | | | | | | | | | | | |
| 0 | 2.7 | 12.6 | 33.1 | 2.9 | 12.8 | 31 | 3 | 12.1 | 37.2 | 2 | 11.5 |
| 10 | 2.7 | 17.5 | 32.3 | 2.9 | 18.4 | 30.4 | 2.9 | 15.5 | 36.8 | 2.1 | 14.7 |
| 20 | 2.7 | 27.5 | 30.6 | 2.8 | 26.9 | 28.9 | 2.7 | 21.1 | 35.2 | 2 | 22.3 |
| 30 | 2.6 | 35.3 | 28.3 | 2.7 | 35.9 | 27 | 2.5 | 30.3 | 32.9 | 2 | 27.7 |
| 40 | 2.4 | 39.6 | 25.2 | 2.8 | 44.2 | 24.3 | 2.3 | 38.7 | 29.5 | 1.9 | 32.9 |
| 50 | 2.2 | 49.3 | 21.8 | 2.3 | 50.9 | 21.4 | 2.1 | 47.2 | 25.6 | 1.8 | 37.1 |
| 60 | 2 | 55.2 | 18.1 | 2 | 56.9 | 18 | 1.8 | 55.3 | 21.2 | 1.7 | 40.8 |
| 70 | 1.7 | 61 | 16.2 | 1.7 | 62.4 | 14.5 | 1.7 | 67.7 | 16.9 | 1.6 | 43.1 |
| 80 | 1.8 | 64 | 11.9 | 1.5 | 63.8 | 12.4 | 1.7 | 68.6 | 14.4 | 1.4 | 44.8 |
| 90 | 1.8 | 65 | 12 | 1.5 | 64.7 | 11 | 1.5 | 69.1 | 12.1 | 1.4 | 45.1 |

Patient 2

| | | | | | | | | | | | |
|---|---|---|---|---|---|---|---|---|---|---|---|
| Maxillary | | | | | | | | | | | |
| 0 | 2.9 | 15.5 | 52.3 | 2.1 | 15.0 | 54.6 | 4.9 | 42.2 | 51.7 | 5.3 | 13.3 |
| 10 | 2.3 | 20.5 | 56.7 | 2.1 | 18.3 | 52.5 | 4.6 | 41.1 | 53.3 | 5.5 | 16.6 |
| 20 | 2.2 | 38.5 | 53.3 | 2.0 | 29.2 | 49.2 | 4.2 | 62.6 | 52.9 | 5.3 | 21.8 |
| 30 | 2.2 | 47.6 | 47.0 | 1.9 | 37.1 | 44.8 | 3.9 | 57.4 | 50.3 | 5.1 | 29.6 |
| 40 | 2 | 70.4 | 46.1 | 1.7 | 42.8 | 39.6 | 3.6 | 64.8 | 45.9 | 4.8 | 36.3 |
| 50 | 2 | 85.6 | 38.4 | 1.5 | 46.9 | 33.6 | 3.3 | 88.2 | 40.5 | 4.4 | 42.2 |
| 60 | 1.8 | 86.1 | 29.0 | 1.5 | 49.9 | 27.0 | 3.1 | 104.5 | 37.2 | 4.1 | 47.6 |
| 70 | 1.4 | 86.5 | 21.1 | 1.3 | 52.1 | 20.1 | 2.9 | 114.6 | 22.8 | 3.8 | 52.6 |
| 80 | 1.4 | 87.4 | 12.9 | 1.1 | 53.6 | 13.8 | 2.6 | 116.1 | 15.5 | 3.6 | 57.5 |
| 90 | 1.6 | 88.3 | 21.0 | 1.1 | 54.5 | 8.9 | 2.4 | 112.7 | 14.4 | 3.3 | 57.6 |
| Mandibular | | | | | | | | | | | |
| 0 | 1.9 | 14.2 | 44.5 | 2.5 | 9.6 | 24.6 | 2.4 | 14.9 | 21.5 | 1.7 | 12.5 |
| 10 | 1.9 | 19.7 | 43.6 | 2.2 | 12.1 | 26.5 | 2.4 | 18.9 | 21.2 | 1.8 | 15.5 |
| 20 | 1.8 | 32.6 | 42.0 | 2.0 | 21.4 | 25.2 | 2.3 | 25.5 | 20.2 | 1.7 | 19.1 |
| 30 | 1.7 | 43.3 | 39.4 | 1.8 | 30.3 | 23.1 | 2.2 | 31.4 | 18.7 | 1.6 | 22.9 |
| 40 | 1.6 | 51.6 | 35.9 | 1.7 | 38.5 | 20.4 | 2.0 | 35.3 | 16.6 | 1.5 | 26.6 |
| 50 | 1.5 | 57.9 | 31.4 | 1.5 | 45.6 | 17.2 | 1.8 | 38.1 | 14.0 | 1.5 | 30.2 |
| 60 | 1.3 | 62.7 | 26.0 | 1.4 | 51.4 | 13.7 | 1.6 | 39.8 | 11.2 | 1.4 | 33.2 |
| 70 | 1.2 | 66.3 | 19.9 | 1.4 | 55.7 | 10.0 | 1.4 | 41.8 | 8.4 | 1.4 | 35.6 |
| 80 | 1.2 | 68.9 | 13.0 | 1.3 | 58.3 | 6.5 | 1.2 | 53.8 | 5.5 | 1.2 | 37.0 |
| 90 | 1.1 | 70.5 | 9.6 | 1.0 | 59.0 | 4.4 | 1.1 | 59.6 | 4.4 | 1.0 | 37.4 |

| | First Premolar | Second Premolar | | | First Molar | | | Second Molar | | |
|---|---|---|---|---|---|---|---|---|---|---|
| Angle | ZX | X | YZ | ZX | XY | YZ | ZX | XY | YZ | ZX |

Patient 1

| | | | | | | | | | | |
|---|---|---|---|---|---|---|---|---|---|---|
| Maxillary | | | | | | | | | | |
| 0 | 44.4 | 6.8 | 19.8 | 61.3 | 16.8 | 17.8 | 44.4 | 14 | 10.7 | 45.7 |
| 10 | 41 | 6 | 22.5 | 60.6 | 16.5 | 15.3 | 43 | 13.7 | 16.1 | 45.5 |
| 20 | 37.2 | 5.5 | 32.3 | 57.8 | 16.3 | 15.5 | 40.3 | 13.3 | 21.8 | 43.7 |
| 30 | 33.2 | 5.2 | 42.3 | 54.1 | 15.5 | 18.4 | 36.7 | 12.8 | 27.8 | 41.4 |
| 40 | 28.9 | 4.8 | 55.8 | 48.5 | 14.3 | 22.3 | 32.5 | 12.2 | 33.5 | 37.9 |
| 50 | 24.6 | 4.3 | 64.1 | 42.5 | 13.1 | 26.3 | 28.3 | 11.6 | 38.2 | 34.1 |
| 60 | 20.5 | 3.7 | 72.4 | 35.9 | 11.8 | 32.6 | 23.9 | 11 | 42.3 | 31.2 |
| 70 | 18 | 3.2 | 80.8 | 29.8 | 10.7 | 38.6 | 20.5 | 10.7 | 45 | 26.6 |
| 80 | 15.5 | 2.9 | 87 | 24.6 | 9.9 | 45.3 | 19.3 | 10.7 | 46.9 | 24.7 |
| 90 | 15.3 | 2.7 | 87.7 | 22.3 | 9.7 | 50.9 | 19 | 11.1 | 47.3 | 24.4 |
| Mandibular | | | | | | | | | | |
| 0 | 33.1 | 3 | 11.6 | 39.9 | 5.8 | 9 | 23.1 | 6.6 | 14.3 | 25 |
| 10 | 33 | 2.9 | 19 | 39.8 | 5.7 | 12.4 | 23.4 | 6.6 | 19.3 | 25 |
| 20 | 31.8 | 2.8 | 25.4 | 38.5 | 5.6 | 17 | 23.1 | 6.6 | 22.2 | 24.6 |
| 30 | 29.9 | 2.5 | 31.7 | 37.6 | 5.5 | 21.3 | 22 | 6.5 | 28.9 | 23.9 |
| 40 | 26.8 | 2.3 | 37.1 | 34.9 | 5.3 | 24.8 | 20.2 | 6.3 | 34.5 | 22.5 |
| 50 | 23.3 | 2.3 | 41.3 | 30.9 | 5.3 | 28.1 | 17.7 | 6 | 38.2 | 20.6 |
| 60 | 19.1 | 1.6 | 44.8 | 26 | 5.2 | 30.2 | 14.8 | 5.8 | 40.9 | 18.5 |
| 70 | 15.2 | 1.6 | 46.8 | 20.5 | 5.2 | 31.5 | 11.7 | 5.6 | 42.2 | 17.2 |
| 80 | 12.7 | 1.7 | 48 | 14.9 | 5.3 | 31.5 | 8.7 | 5.5 | 42.5 | 14.7 |
| 90 | 12.2 | 1.7 | 47.9 | 13.1 | 5.6 | 31.8 | 6.6 | 5.5 | 41.8 | 13.9 |

Patient 2

| | | | | | | | | | | |
|---|---|---|---|---|---|---|---|---|---|---|
| Maxillary | | | | | | | | | | |
| 0 | 40.5 | 3.1 | 18.3 | 56 | 19.5 | 9.2 | 45.2 | 16.2 | 15.3 | 33.8 |
| 10 | 33.8 | 3.3 | 28.4 | 59.9 | 18.3 | 15.6 | 44.2 | 15.6 | 14.7 | 33.0 |
| 20 | 31.6 | 3.2 | 42 | 54.5 | 16.6 | 22.1 | 43.7 | 14.9 | 15.8 | 32.7 |
| 30 | 29.4 | 3.0 | 50.4 | 51.9 | 14.7 | 29.7 | 42.9 | 14.1 | 18.1 | 31.1 |
| 40 | 27 | 2.8 | 68.7 | 48.2 | 12.9 | 39.2 | 39.5 | 13.2 | 21.2 | 28.9 |
| 50 | 24.3 | 2.5 | 70.2 | 44 | 11.2 | 42 | 39.1 | 12.2 | 24.7 | 25.7 |
| 60 | 21.4 | 2.2 | 68 | 39.1 | 9.2 | 48 | 37.4 | 11.3 | 27.8 | 24.3 |
| 70 | 18.2 | 2.1 | 75 | 33.9 | 10.8 | 50.1 | 35.6 | 10.8 | 31.0 | 17.7 |
| 80 | 14.8 | 1.8 | 88.7 | 28.4 | 8.3 | 53.7 | 27.1 | 9.8 | 33.2 | 19.1 |
| 90 | 11.5 | 1.6 | 88.8 | 23 | 7.3 | 55.5 | 18.8 | 8.7 | 35.6 | 16.8 |
| Mandibular | | | | | | | | | | |
| 0 | 31.9 | 1.8 | 11.2 | 39.8 | 5.0 | 10.3 | 22.3 | 4.5 | 19.6 | 1.9 |
| 10 | 31.4 | 1.6 | 15 | 41.7 | 4.9 | 11.3 | 21.6 | 3.7 | 25.5 | 1.9 |
| 20 | 30.2 | 1.6 | 18.9 | 42.6 | 4.8 | 12.2 | 20.7 | 3.2 | 28.5 | 1.8 |
| 30 | 28.5 | 1.5 | 24.5 | 39.5 | 4.6 | 13.5 | 19.5 | 3.0 | 30.5 | 1.7 |
| 40 | 26.2 | 1.3 | 38.6 | 34.9 | 4.4 | 19.2 | 17.7 | 3.0 | 31.7 | 1.6 |
| 50 | 23.3 | 1.2 | 40.2 | 29.9 | 4.2 | 20.0 | 15.3 | 2.8 | 32.0 | 1.5 |
| 60 | 20.1 | 1.1 | 40.3 | 23.8 | 4.0 | 23.0 | 12.8 | 2.4 | 32.0 | 1.3 |
| 70 | 16.8 | 1.2 | 45.5 | 17.9 | 3.9 | 25.2 | 9.8 | 2.7 | 32.4 | 1.2 |
| 80 | 8.6 | 1.1 | 50.5 | 12.7 | 3.8 | 26.1 | 7.4 | 2.9 | 33.3 | 1.2 |
| 90 | 7.2 | 1.2 | 56.5 | 12.7 | 3.9 | 27.3 | 6.9 | 2.2 | 35.2 | 1.1 |

TABLE 11 shows a percent variation of k on each plane varying the force direction from one coordinate axis to the other.

TABLE 11

| | | | | | | |
|---|---|---|---|---|---|---|
| Central | Lateral | | First | Second | First | Second |
| incisor | Incisor | Canine | premolar | premolar | molar | molar |

Patient 1

| | | | | | | | |
|---|---|---|---|---|---|---|---|
| Maxillary | | | | | | | |
| Plane XY | 29 | 42 | 90 | 79 | 151 | 73 | 26 |
| Plane YZ | 342 | 366 | 450 | 310 | 342 | 185 | 342 |
| Plane ZX | 126 | 189 | 174 | 190 | 174 | 133 | 87 |
| Mandibular | | | | | | | |
| Plane XY | 48 | 96 | 98 | 49 | 71 | 3.4 | 20 |
| Plane YZ | 415 | 403 | 471 | 291 | 312 | 251 | 192 |
| Plane ZX | 175 | 181 | 207 | 170 | 206 | 248 | 80 |

Patient 2

| | | | | | | | |
|---|---|---|---|---|---|---|---|
| Maxillary | | | | | | | |
| Plane XY | 107.1 | 90.9 | 104.2 | 66.7 | 106.3 | 167.1 | 86.2 |
| Plane YZ | 469.6 | 263.3 | 179.6 | 333.1 | 385.3 | 503.2 | 142.1 |
| Plane ZX | 339.5 | 513.4 | 270.1 | 252.2 | 160.4 | 140.4 | 101.2 |
| Mandibular | | | | | | | |
| Plane XY | 72.7 | 150 | 118.1 | 80 | 63.6 | 145 | 104.5 |
| Plane YZ | 396.5 | 513.5 | 300 | 199.2 | 404.4 | 165.1 | 79.6 |
| Plane ZX | 363.5 | 502.2 | 388.6 | 343 | 235.4 | 223.2 | 158.1 |

TABLE 12
Center of Resistance

| | Predicted | Calculated |
|---|---|---|
| Loc x (mm) | 0.9 | 0.5 |
| Loc y (mm) | −0.1 | −0.1 |
| Loc z (mm) | −5.2 | −5.5 |

K

| | | |
|---|---|---|
| Angle (°) | Predicted K | Calculated K |

Plane XY

| | | |
|---|---|---|
| 0 | 2.9 | 3.4 |
| 10 | 2.8 | 3.4 |
| 20 | 2.6 | 3.3 |
| 30 | 2.5 | 3.1 |
| 40 | 2.4 | 2.9 |
| 50 | 2.3 | 2.7 |
| 60 | 2.2 | 2.4 |
| 70 | 2.0 | 2.1 |
| 80 | 1.9 | 1.8 |
| 90 | 1.8 | 1.5 |

Plane YZ

| | | |
|---|---|---|
| 0 | 14.3 | 9.2 |
| 10 | 20.7 | 15.3 |
| 20 | 27.2 | 19.5 |
| 30 | 33.6 | 24.3 |
| 40 | 40.0 | 31.4 |
| 50 | 46.4 | 42.3 |
| 60 | 52.8 | 45.8 |
| 70 | 59.3 | 49.5 |
| 80 | 65.7 | 55.4 |
| 90 | 72.1 | 62.2 |

Plane ZX

| | | |
|---|---|---|
| 0 | 37.5 | 35.7 |
| 10 | 34.0 | 35.2 |
| 20 | 30.5 | 33.9 |
| 30 | 26.9 | 31.9 |
| 40 | 23.4 | 29.2 |
| 50 | 19.9 | 24.1 |
| 60 | 16.3 | 21.0 |
| 70 | 12.8 | 17.5 |
| 80 | 9.3 | 14.9 |
| 90 | 5.7 | 13.3 |

In some embodiments, a random mandibular central incisor can be selected to demonstrate the predictor efficacy. TABLE 12 and FIG. 7B show k datasets obtained on a random mandibular central incisor, respectively, through FEM and morphological predictors, so that the statistical model accuracy can be compared to the FEM.

FIG. 9E shows an example process/method 990 flow for a full treatment simulation in accordance with principles of the present disclosure. In some embodiments, as shown in FIG. 9E, information, such as information related to the aligners/attachments, teeth, PDL, bone sockets, etc., can be dynamically and/or iteratively provided to a simulation model 991, e.g., including FEA as discussed in various embodiments/aspects throughout this disclosure, to generate and provide information related to the deformed positions of the teeth, bone sockets, etc., as well as information for changes to or additional aligners/attachments, for the various treatment steps. The simulation of the full treatment may allow/facilitate changing of the attachment on the teeth during treatment, as well as changing the aligner features for the various treatment steps. In one example, a relatively soft aligner material can be used in the initial/first treatment steps and materials having other, varying hardness properties (e.g., successively harder materials) can be used for subsequent steps.

For example, FIG. 9E shows that, at step 992, data or information related to an initial aligner 992A, data/information related to the teeth, PDL, bone 992B, e.g., information related to initial positons, orientations, etc. thereof, and/or information related to an initial attachment 992C are provided to a model/simulator 991, which employs FEA, statistical methods, as provided by embodiments of the present disclosure. The initial data/information related to the aligner 992A, information related to the teeth, PDL, bone 992B, and/or information related to an attachment 992C can be gathered using the processes/methods described herein, such as being obtained from images, 3D models, etc. It also will be understood that the initial information can be obtained/retrieved using any suitable methods, processes, mechanisms, etc. In step 992, the model/simulator 991 is applied to the initial data/information 992A, 992B, 992C to generate information/data, such as information related to the position of the deformed teeth, bone sockets, etc. due the application of the initial aligner in step 992, as well as information for aligner/attachments updates or changes.

Subsequently, at step 994, the model/simulator 991 can provide/export information from the simulation/model in step 992, e.g., the information developed with regard to the deformed/final teeth position 994A by the application of the initial aligner in step 992. Furthermore, data information related to a new 0.2 mm uniform PDL around each tooth 994B can be generated/recreated, and information/data related to the new socket for teeth and PDL on the original bone 994C also can be generated/recreated. In some examples, the tooth and PDL positions is updated according to stress and strain values of bone, PDL, and tooth, e.g., to simulate osteogenesis and osteonecrosis processes.

In step 994 in FIG. 9E, the information/data related to the deformed/final teeth position 994A, the 0.2 mm uniform PDL data 994B, and new bone sockets data 994C, as well as a next aligner or attachment 994D including any updates or changes can be uploaded to the model/simulator 991 to determine the information for the next step n, which will generally be the same as step 994, and the process repeats following the same workflow shown in FIG. 9E, until the end of treatment, e.g., until the last aligner/attachment or the selected tooth or group of teeth reach a desired position, orientation, configuration, etc.

Furthermore, in some embodiments, it may not always be practical for dental professionals to run an FEM model before each and every orthodontic treatment, e.g. at each treatment step, and thus, it is possible to evaluate an approximate attachment configuration without FEA, which could be useful to optimize the treatment quickly (e.g., the following can be a part of the model/simulator 991 as shown in FIG. 9E, and can be applied when the full FEA is not desired).

For example, it can be recognized that the k factor illustrates that the tooth morphology is related to the distance C_{RES}-C_{ROT}, which can describe the quality of the movement on each plane. The quality also can be related to the attachment shape and depth and the expected movement and to the attachment location on the crown surface in relation to the Axis of resistance. The quantity of the movement further can be related to the attachment dimensions (such as diameter and depth) and to the amount of activation of the aligner for each movement. The quantity of load delivered to each tooth can be controlled to match the specific tooth resistance number. Also for crown movements the maximum movement must be limited according to the PDL thickness which is dependent on tooth morphology and age. Moreover, the attachments size generally does not provide only benefits, but the larger the attachment also increases the discomfort for the patient. Also, if the attachment is too large, it can be difficult to remove the aligner.

The quality of the movement can be defined not only by the C_{ROT} location but also by the translation or rotation axis that can be calculated directly using the measure force system delivered to the tooth.

Considering this, it is possible to create a ranking of all configurations and to use a formula as follows to assign a point to each scenario:

P=A*Quality+B*Quantity−Clinical Factor Eq. 9

A and B and Clinical factor (durability and ease of removal) can include a weight that can be assigned depending on how much significance each parameter is given/assigned in the specific situation. Generally, but not always, A+B+Discomfort=1 (100%), and thus, the percentage weight for each factor may be known/can be determined. Furthermore, it can be understood that the quantity of movements depends on the attachment diameter, depth, and/or specific shape, which generates the necessary grip depending on the movement, e.g., (depth*D+Diameter*E+shape_factor)->Quantity. It also can be known that the quality depends on K and the shape, e.g., (kxy*shape+kyz*shape+kzx*shape_factor)->Quality. Clinical factor can be affected by the attachment size, and thus, while the depth of the attachment may increase grip, it also can decrease its durability and the aligner's ease of removal.

Depth and diameter can affect also the quality. Moreover, the attachment location and orientation on the crown surface also affect the quality of movement.

It also is possible to assign a point to each configuration, from Eq. 9, as follows:

P=A*(kxy*shape+kyz*shape+kzx*shape_factor)+B*(depth*D+Diameter*E+shape_factor)−C*(depth*F+diameter*G+volume_encumbrance) Eq. 10

A; B; C; D; E; F; G; shape_factor generally depend on the movement and the specific weight that the user prefers to assign to each parameter. As an example, if patient discomfort is not a concern, a low value at C can be assigned. A, B, and C further can be user dependent and each user can decide if whether they care more about quality; quantity or patient comfort. As with equation 9, a generally, but not always, A+B+C=1 (100%), and so, the percentage weight for each factor may be known. D, E, F, G may depend on tooth morphology and movement and can be calculated running FEA analysis on multiple teeth and movement. Volume_encumbrance can depend on the shape and size of the attachment, but is different than shape_factor, e.g., Volume_encumbrance can be related to how difficult it makes to remove the aligner. The shape factor can depend on the movement and the spatial plane. As shown in the demonstration of k efficacy, the attachment generally should provide the correct grip, with sharp edges on the specific plane depending on the movement.

FIG. 10 shows a process or method 1000 for simulation of aligners and/or attachments for a patient according to one embodiment of the present disclosure. As shown in FIG. 10, at step 1002, information for a patient's group teeth can be collected/gathered, e.g., using a CBCT and optimal scanner to obtain one or more images or other information related to a patient's group of teeth. At step 1004, an aligner is designed using CAD modelling. At Step 1006, input from steps 1002 and 1004 are used to define a coordinate system according to the occlusal plane for each tooth. In step 1008, the vestibular crown center for the tooth that needs to be moved is located. In step 1010, C_{RES} is located using the coefficients in Table 9. For example, the equations for determination of an X coordinate of a maxillary tooth would be:

$$X = 0.733 + MD * ( - 0.285 ) + LB * ( 0.21 ) \qquad Eq . 11$$

where MD is the mesiodistal tooth dimension at the CEJ and LB is the linguo-buccal tooth dimension at the CEJ.

In step 1012, the attachment at the crown center is designed and provided to Steps 1014 and 1016. In these steps, FEA with and without the occlusal loads, respectively, are performed and supplied to a force estimation system. In Step 1018, center of resistance coordinates are calculated or determined using one or more predictors, such as the predictors shown in TABLE 9. In step 1020, the attachments are shape tested. Different basic shapes, for example, triangular, cylindrical, and cubic were tested with FEA. These shapes are non-limiting examples as many possible geometrical designs can be tested until the results are satisfactory. If the attachments meet the testing criteria, then the different shapes are compared in Step 1024 according to the methods disclosed herein. In step 1026, for the most effective shape, the model is evaluated with respect to different diameter and depth. The force systems measured for each shape are compared and a numerical value (ranking) is assigned to describe the effectiveness of the proposed model:

Value=QI*(A*C_{ROT}+B*Axis)+Qt*Load+Clinical Factors Eq. 12

Where QI is a qualitative index, C_{ROT} and Axis are Center of resistance to ideal center of resistance and deviation of movement axis to the ideal movement axis, respectively, Qt is a quantitative index, and the clinical factors, which include without limitations, durability and ease of removal.

FIG. 9C represents the steps to simulate the different shapes. Further, the comparison/ranking is evaluated based on quantity, quality (measured in terms of distances) and clinical factors that can have a different weight depending on the desired movement.

With respect to the diameter, the maximum value depends on the crown size. In certain embodiments, the diameters can range from 1 mm to 4 mm. In certain embodiments, the depth can range from 0.75 mm to 2 mm. In an embodiment, once the optimal shape is defined, all the diameters and depths are tested to find the best combination. In other embodiments, using the results as illustrated in FIGS. 6A-6Q, which are representative only for one tooth, it is possible to choose depth and diameter knowing that they affect differently the quantity and quality of the movement and therefore depending on the desired movement quality or quantity it can be decided to combine diameter, depth and position. In step 1028, for the most effective combination of diameter and depth, the model is evaluated with respect to with different positions on the crown, while moving the attachment horizontally and vertically. In the next step 1030, these different configurations are tested according to the methods disclosed herein (such as that illustrated in FIG. 9C) and in step 1032, the most effective configuration is selected for design of the aligner/attachment. This step 1030 can be also referred to as ranking. In certain embodiments, the ranking can be determined by use of Eq. 12. In certain embodiments, the ranking can be determined by use of Eq. 9 or Eq. 12, or a combination thereof. All the collected force systems are utilized to calculate (a) force and moments (desired and undesired directions), (b) center of rotation distance from the ideal center of rotation using k and the force systems and applying the equations 6, 7, and 8, (c) axis of rotation deviation from the ideal axis of rotation using moments in case of rotational movements and forces in case of translation movements and calculating the direction of the resulting moment or force, wherein the difference between the calculated direction and the ideal movement direction can be calculated by subtraction, and (d) Volume and shape factor from the design of the attachment. With all these results, the tested configurations are ranked for the optimal attachment or aligner configuration.

FIG. 11 illustrates a set of components of an example computing device 1100 that can be utilized to implement aspects of the various embodiments. Such a computing device 1100 may be used as medical equipment 218A in FIG. 2A, in a professional environment, when provided with the software to configure it to perform the methods discussed in FIGS. 9A, 9B, 9C, 9D, 9E, and 10. In this example, the device 1100 includes at least one processor 1102 for executing instructions that can be stored in a memory device or element 1104. As would be apparent to one of ordinary skill in the art, the device can include many types of memory, data storage or computer-readable media, such as a first data storage for program instructions for execution by the at least one processor 1102, the same or separate storage can be used for images or data, a removable memory can be available for sharing information with other devices, and any number of communication approaches can be available for sharing with other devices. The device 1100 may include at least one type of display element 1106, such as a touch screen, electronic ink (e-ink), organic light emitting diode (OLED) or liquid crystal display (LCD), although devices such as servers might convey information via other means, such as through a system of lights and data transmissions. The device 1100 typically will include one or more networking components 1114, such as a port, network interface card, or wireless transceiver that enables communication over at least one network. The device 1100 can include at least one input element 1118 able to receive conventional input from a user. This conventional input can include, for example, a push button, touch pad, touch screen, wheel, joystick, keyboard, mouse, trackball, keypad or any other such device or element whereby a user can input a command to the device. These I/O devices for the input element 1118 could even be connected by a wireless infrared or Bluetooth or other link as well in some embodiments. In some embodiments, however, such a device might not include any buttons at all and might be controlled only through a combination of visual and audio commands such that a user can control the device 1100 without having to be in contact with the device.

The device 1100 may also include one or more imaging elements 1112 for performing the scans and capturing images as required-including using a CBCT and an optical scanner. One or more orientation elements 1108 may be used to determine the orientation of the device, for example in relation to a user's face or eyes. Various camera-based and other sensors, as part of the imaging element 1112, may be used to determine orientation. An orientation element 1108 can determine the position of the device. The orientation element 1108 can use one or more of GPS, local network detection, Bluetooth connection, or other protocols. One or more input elements 1118 can register user input, for example input received from a touch screen display. An example device 1100 will also include power components 1116 and wireless ability in network components 1114 to communicate with other devices wirelessly.

Devices like device 1100 can also include a computer-readable storage media reader, a communications device (e.g., a modem, a network card (wireless or wired), an infrared communication device) and working memory as described above. The computer-readable storage media reader can be connected with, or configured to receive, a computer-readable storage medium representing remote, local, fixed and/or removable storage devices as well as storage media for temporarily and/or more permanently containing, storing, transmitting and retrieving computer-readable information. The system and various devices also typically will include a number of software applications, modules, services or other elements located within at least one working memory device, including an operating system and application programs such as a client application or Web browser. It should be appreciated that alternate embodiments may have numerous variations from that described above. For example, customized hardware might also be used and/or particular elements might be implemented in hardware, software (including portable software, such as applets) or both. Further, connection to other computing devices such as network input/output devices may be employed.

Device 1100 also includes classifier 1110 to perform the simulation and prediction aspects discussed with regards to the methods in FIGS. 9A, 9B, 9C, 9D, and 9E. Alternatively, Device 1100 shares some functions with a network server, which is discussed in FIG. 11. The network server then handles part of the high load processing for prediction and simulating models using the FEA, for example. As such, when a subject's set of teeth is captured via an imaging element 1112, the images may be parsed in the device 1100 or shared with the network server. The tooth measurement data from the images may be extracted and used to build the tooth models discussed with respect to sub-processes 982A, 982B, in FIG. 9D. That data may be then provided to the device 1100 for processing in the classifier 1110 using a simulation model such as the FEA. Such a process avoids latency of high load data transfer and efficiently uses the resources available to the professional to generate an aligner than incorporates the long term design guidelines provided in the present disclosure.

FIG. 12 illustrates an example system environment 1200 that can be used to implement aspects of various embodiments. The illustrative environment 1200 includes at least one application server 1208, a web server 1210, and a data store 1212. It should be understood that there can be several application servers, layers or other elements, processes or components, which may be chained or otherwise configured, which can interact to perform tasks such as obtaining data from an appropriate data store. As used herein, the term “data store” refers to any device or combination of devices capable of storing, accessing and retrieving data, which may include any combination and number of data servers, databases, data storage devices and data storage media, in any standard, distributed or clustered environment. The application server 1208 can include any appropriate hardware and software for integrating with the data store as needed to execute aspects of one or more applications for the client device and handling a majority of the data access and business logic for an application. The application server 1208 provides access control services in cooperation with the data store and is able to generate content such as text, graphics, audio and/or video to be transferred to the user, which may be served to the user by the Web server in the form of HTML, XML or another appropriate structured language in this example. The handling of all requests and responses, as well as the delivery of content between the client device and the application server, can be handled by the Web server 1210. It should be understood that the Web server 1210 and application servers 1208 are merely example components, as structured code discussed herein can be executed on any appropriate device or host machine as discussed elsewhere herein.

The data store 1212 can include several separate data tables, databases or other data storage mechanisms and media 1214, 1216, 1218 for storing data relating to a particular aspect. For example, the data store 1212 illustrated includes mechanisms for storing content such as a data storage, session information storage, and a classifier 1214, 1216, 1218. The session information may correspond to user and profile information, which can be used to serve content for the production side. The data store 1212 is also shown to include the session information mechanism 1216 for storing log or session data. It should be understood that there can be many other aspects that may need to be stored in the data store, such as page image information and access rights information, which can be stored in any of the above listed mechanisms as appropriate or in additional mechanisms in the data store. The data store 1212 is operable, through logic associated therewith, to receive instructions from the application server and obtain, update or otherwise process data in response thereto. In one example, a user might submit a search request for a certain type of item. In this case, the data store 1212 might access the user information to verify the identity of the user and can access the catalog detail information to obtain information about items of that type. The information can then be returned to the user, such as in a results listing on a Web page that the user is able to view via a browser on the user device. Information for a particular item of interest can be viewed in a dedicated page or window of the browser.

Each server 1210, 1208 typically will include an operating system that provides executable program instructions for the general administration and operation of that server and typically will include computer-readable medium storing instructions that, when executed by a processor of the server, allow the server to perform its intended functions. Suitable implementations for the operating system and general functionality of the servers are known or commercially available and are readily implemented by persons having ordinary skill in the art, particularly in light of the disclosure herein.

The environment 1200 in one embodiment is a distributed computing environment utilizing several computer systems and components that are interconnected via communication links, using one or more computer networks or direct connections. However, it will be appreciated by those of ordinary skill in the art that such a system could operate equally well in a system having fewer or a greater number of components than are illustrated. Thus, the depiction of the systems herein should be taken as being illustrative in nature and not limiting to the scope of the disclosure.

The various embodiments can be further implemented in a wide variety of operating environments, which in some cases can include one or more user computers or computing devices 1202 and 1204 which can be used to operate any of a number of applications. User or client devices 1202 and 1204 can include any of a number of general purpose personal computers, such as desktop or laptop computers running a standard operating system, as well as cellular, wireless and handheld devices running mobile software and capable of supporting a number of networking and messaging protocols. Such a system can also include a number of workstations running any of a variety of commercially-available operating systems and other known applications for purposes such as development and database management. These devices can also include other electronic devices, such as dummy terminals, thin-clients, gaming systems and other devices capable of communicating via a network.

Most embodiments utilize at least one network 1206 that would be familiar to those skilled in the art for supporting communications using any of a variety of commercially-available protocols, such as TCP/IP, FTP, UPnP, NFS, and CIFS. The network 1206 can be, for example, a local area network, a wide-area network, a virtual private network, the Internet, an intranet, an extranet, a public switched telephone network, an infrared network, a wireless network and any combination thereof.

In embodiments utilizing a Web server 1210, the Web server can run any of a variety of server or mid-tier applications, including HTTP servers, FTP servers, CGI servers, data servers, Java servers and business application servers. The server(s) may also be capable of executing programs or scripts in response requests from user devices, such as by executing one or more Web applications that may be implemented as one or more scripts or programs written in any programming language, such as Java®, C, C # or C++ or any scripting language, such as Perl, Python or TCL, as well as combinations thereof. The server(s) may also include database servers, including without limitation those commercially available from Oracle®, Microsoft®, Sybase® and IBM® as well as open-source servers such as MySQL, Postgres, SQLite, MongoDB, and any other server capable of storing, retrieving and accessing structured or unstructured data. Database servers may include table-based servers, document-based servers, unstructured servers, relational servers, non-relational servers or combinations of these and/or other database servers.

The environment 1200 can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (SAN) familiar to those skilled in the art. Similarly, any necessary files for performing the functions attributed to the computers, servers or other network devices may be stored locally and/or remotely, as appropriate. Where a system includes computerized devices, each such device can include hardware elements that may be electrically coupled via a bus, the elements including, for example, at least one central processing unit (CPU), at least one input device (e.g., a mouse, keyboard, controller, touch-sensitive display element or keypad) and at least one output device (e.g., a display device, printer or speaker). Such a system may also include one or more storage devices, such as disk drives, magnetic tape drives, optical storage devices and solid-state storage devices such as random access memory (RAM) or read-only memory (ROM), as well as removable media devices, memory cards, flash cards, etc.

Storage media and other non-transitory computer readable media for containing code, or portions of code, can include any appropriate media known or used in the art, such as but not limited to volatile and non-volatile, removable and non-removable media implemented in any method or technology for storage of information such as computer readable instructions, data structures, program modules or other data, including RAM, ROM, EEPROM, flash memory or other memory technology, CD-ROM, digital versatile disk (DVD) or other optical storage, magnetic cassettes, magnetic tape, magnetic disk storage or other magnetic storage devices or any other medium which can be used to store the desired information and which can be accessed by a system device. Based on the disclosure and teachings provided herein, a person of ordinary skill in the art will appreciate other ways and/or methods to implement the various embodiments.

As discussed, different approaches can be implemented in various environments in accordance with the described embodiments. As will be appreciated, although a Web-based environment is used for purposes of explanation in several examples presented herein, different environments may be used, as appropriate, to implement various embodiments. The system includes an electronic client device, which can include any appropriate device operable to send and receive requests, messages or information over an appropriate network and convey information back to a user of the device. Examples of such client devices include personal computers, cell phones, handheld messaging devices, laptop computers, set-top boxes, personal data assistants, electronic book readers and the like. The network can include any appropriate network, including an intranet, the Internet, a cellular network, a local area network or any other such network or combination thereof. Components used for such a system can depend at least in part upon the type of network and/or environment selected. Protocols and components for communicating via such a network are well known and will not be discussed herein in detail. Communication over the network can be enabled via wired or wireless connections and combinations thereof. In this example, the network includes the Internet, as the environment includes a Web server for receiving requests and serving content in response thereto, although for other networks, an alternative device serving a similar purpose could be used, as would be apparent to one of ordinary skill in the art.

It will, however, be evident that various modifications and changes may be made thereunto without departing from the broader spirit and scope of the embodiments as set forth in the claims.

What is claimed is:

1. A system for simulation of dental aligners or attachments configured to move one or more teeth of a group of teeth of a patient to a selected configuration, comprising:
at least one processor; and
memory comprising instructions that, when executed in part by the at least one processor, cause the at least one processor to:
determine axial plane values for the group of teeth of the patient;
determine a coordinate system based at least in part on the determined axial plane values;
determine a crown location for at least one tooth of the group of teeth using the coordinate system;
determine an attachment at the crown location for a digital aligner for the group of teeth;
generate a digital penetration between the digital aligner and the at least one tooth;
determine, by at least a finite element analysis, orthodontic data for a center of rotation of the at least one tooth based at least in part on the digital penetration; and
develop at least one variation to the attachment or the digital aligner based in part on the orthodontic data.

2. The system of claim 1, wherein the instructions that, when executed in part by the at least one processor, further cause the at least one processor to:
generate a physical aligner or attachment from the digital aligner with the at least one variation.

3. The system of claim 1, wherein the instructions that, when executed in part by the at least one processor, further cause the at least one processor to:
calculate the center of rotation for the tooth based at least in part on the digital penetration.

4. The system of claim 1, wherein the instructions that, when executed in part by the at least one processor, further cause the at least one processor to:
generate a digital view of the digital aligner in an initial configuration.

5. The system of claim 1, wherein the instructions that, when executed in part by the at least one processor, further cause the at least one processor to:
generate a new digital view of the digital aligner in new configuration after application of the at least one variation.

6. The system of claim 1, wherein the at least one variation is a shape or a material for a physical aligner corresponding to the digital aligner.

7. The system of claim 1, wherein the instructions that, when executed in part by the at least one processor, further cause the at least one processor to:
apply one or more of a Cone Beam Computed Tomography (CBCT) and an optical scan for a subject; and
determine the axial plane values for at least the group of teeth from the one or more of the CBCT and the optical scan.

8. The system of claim 1, wherein the instructions that, when executed in part by the at least one processor, further cause the at least one processor to:
generate averaged dimensioned teeth for a subject; and
determine the axial plane values from the averaged dimensioned teeth.

9. The system of claim 1, wherein the instructions that, when executed in part by the at least one processor, further cause the at least one processor to:
determine the attachment at the crown location for the digital aligner using Boolean operations.

10. A method for simulation of dental aligners or attachments configured to move one or more teeth of a group of teeth of a patient to a selected configuration, comprising:
determining axial plane values for a group of teeth;
determining a coordinate system from the axial plane values;
locating a crown location for a tooth of the group of teeth using the coordinate method;
determining an attachment at the crown location for a digital aligner for the group of teeth;
generating a digital penetration between the digital aligner and the tooth;
determining, by at least a finite element analysis, orthodontic data for a center of rotation for the tooth based at least in part on the digital penetration; and
providing at least one variation to the attachment or the digital aligner based in part on the orthodontic data.

11. The method of claim 10, further comprising:
generating a physical aligner from the digital aligner with the at least one variation.

12. The method of claim 10, further comprising:
calculating the center of resistance for the tooth based at least in part on the digital penetration.

13. The method of claim 10, further comprising:
generating a digital view of the digital aligner in an initial configuration.

14. The method of claim 10, further comprising:
generating a new digital view of the digital aligner in new configuration after application of the at least one variation.

15. The method of claim 10, wherein the at least one variation is a shape or a material for a physical aligner corresponding to the digital aligner.

16. The method of claim 10, further comprising:
applying a Cone Beam Computed Tomography (CBCT) or an optical scan for a subject; and
determining the axial plane values for at least the group of teeth from the CBCT or the optical scan.

17. The method of claim 10, further comprising:
generating averaged dimensioned teeth for a subject; and
determining the axial plane values from the averaged dimensioned teeth.

18. The method of claim 10, further comprising:
determining the attachment at the crown location for the digital aligner using Boolean operations.

19. A method for simulation of one or more attachments or aligners configured to move one or more teeth of a group of teeth of a patient to a selected position or location, comprising:
receiving or obtaining information or data related to the group of teeth of a patient;
generating a digital model based at least in part on the information or data related to the group of teeth;
generating an aligner or an attachment or information related thereto for the group of teeth;
calculating an approximate center of resistance coordinates for the group of teeth using one or more predictors;
calculating an approximate parameter value for the at least one tooth for each plane using one or more predictors; and
generate, using at least a finite element analysis, center of rotation coordinates based at least in part of the parameter value for each plane.

20. A method for simulating a series of treatment steps applying a plurality of aligners or attachments configured to move one or more teeth of a group of teeth of a patient to a selected position or configuration, comprising:
(i) receiving information related to an initial configuration of a group of teeth of the patient;
(ii) generating an aligner or attachment for the initial configuration;
(iii) providing the information related to initial configuration and information related to the aligner or attachment to a finite element model;
(iv) generating information related to a deformed configuration for the group of teeth using the finite element model;
(v) generating at least one additional aligner or attachment for the deformed configuration;
(vi) providing the information related to the deformed configuration and information related to the additional aligner or attachment to the finite element model; and
(vii) repeating steps (iv)-(vi) until one or more teeth of the group of teeth reach the selected position or configuration.
